(19) United States
(12) Patent Application Publication
(10) Pub. No.: US 2013/0059903 A1
(43) Pub. Date: Mar. 7, 2013

(54) Compositions and Methods for Characterizing Breast Cancer

(71) Applicants: Venu Raman, Ellicott City, MD (US); Farhad Vesuna, Baltimore, MD (US)

(72) Inventors: Venu Raman, Ellicott City, MD (US); Farhad Vesuna, Baltimore, MD (US)

(73) Assignee: THE JOHNS HOPKINS UNIVERSITY, Baltimore, MD (US)

(21) Appl. No.: 13/634,315

(22) PCT Filed: Mar. 14, 2011
(86) PCT No.: PCT/US11/28358

§ 371 Date: Nov. 13, 2012

Related U.S. Application Data
(60) Provisional application No. 61/313,340, filed on Mar. 12, 2010.
(60) Provisional application No. 61/313,472, filed on Mar. 12, 2010.
(60) Provisional application No. 61/347,163, filed on May 21, 2010.

(51) Int. Cl. G01N 33/574; A61P 35/00; C40B 30/04; A61K 31/7088; C12Q 1/68; G01N 27/62

(52) U.S. Cl. 514/44 A; 435/7.23; 435/6.11; 435/6.12; 506/9

(57) ABSTRACT

The invention provides compositions and methods for characterizing breast cancer stem In particular, the invention provides for the identification of cells expressing Twist and CD44 that express little or virtually undetectable levels of CD24 (i.e. a Twist⁺/CD44⁺/CD24^{-/low} cell sub-population). The presence of such cells in a breast cancer specimen identifies the breast cancer as having increased metastic potential. Such cancers are identified as requiring aggressive therapies. Accordingly, the invention provides biomarkers suitable for identifying, diagnosing, and monitoring treatment of a subject with breast cancer.

### CROSS-REFERENCE TO RELATED APPLICATION

This application claims the benefit of the following U.S. Provisional Application No's.: 61/313,340, filed Mar. 12, 2010, 61/313,472, filed Mar. 12, 2010, and 61/347,163, filed May 21, 2010, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant No's: R01CA097226, R01CA140226, P50CA103175, MSCRFE-0128-00 and MSCRFF-0005-00. The government has certain rights in the invention.

### BACKGROUND OF THE INVENTION

The conventional view of cancer is that this disease is caused by fast-growing, highly proliferative cells caused by multi-step mutation events at the cellular level. Current treatments are directed towards the eradication of this population of cells, either by chemotherapy or irradiation. In addition, more complex immunotherapies and gene therapies are emerging. All of these methods eradicate a significant volume of the tumor mass by targeting the neoplastic, highly proliferative cells making up the majority of its volume. When these methods fail, it is believed that some cells survived the therapy process, either because of inadequacies in the treatment or because a proportion of the cells were resistant.

The concept that cancers arise from stem cells has recently been given new impetus by advances in stem cell biology. This hypothesis holds that tumors originate from stem cells through dysregulation of the tightly regulated process of self-renewal. Tumors thus retain a subcomponent of cells that retain key stem cell properties, including the ability to self-renew. This drives tumorigenesis and aberrant differentiation. This theory has gained ground among many researchers in this area.

The implications of this hypothesis are wide-ranging and fundamental, having ramifications for cancer risk assessment, early detection, prognostication, and development. It also casts doubt on, and perhaps explains, the failure of current anti-cancer therapeutics that target the end stage differentiated cancer cells rather than the cancer stem cells from which they are derived.

The cancer stem cell hypothesis requires that a rare population of cancer stem cells be targeted. Of course, this approach carries with it an inherent problem, in that the body's healthy stem cell population must be spared. In particular, the stem cell model has important implications for the development of biomarkers for the early detection of cancer, since this model holds that important prognostic and predictive information can be obtained from identifying cancer stem cells when they arise. If biomarkers can be developed that distinguish cancer stem cells from their healthy counterparts, such biomarkers may also be used to target the diseased cells and thus reduce cancer stem cell numbers. Additionally, such biomarkers may also be used to increase the accuracy with which pathologists are able to identify cancer stem cells in biopsy samples. In particular, therapeutic interventions that either induce apoptosis or differentiation with a loss of self-renewal capacity in these cells represents a rational therapeutic approach to cancer treatment.

There is thus a great need for a method that specifically and selectively identifies cancer stem cells, and which differentiates these cancer stem cells from healthy stem cells.

### Conventional

### SUMMARY OF THE INVENTION

The present invention provides compositions and methods for diagnosing and characterizing cancer.

In one aspect, the invention generally provides a method for identifying a subject as having neoplasia having high metastatic potential, the method involves detecting an increase (e.g., about 5%, 10%, 25%, 50%, 75%, 80%, 90%) in the level of Twist and CD44 polypeptides or nucleic acid molecules, and a reduction (e.g., about 5%, 10%, 25%, 50%, 75%, 80%, 90%) in the level of CD24 polypeptides or nucleic acid molecules in a biological sample from the subject relative to levels present in a control, thereby identifying the neoplasia as having high metastatic potential. In one embodiment, the neoplasia is an epithelial carcinoma (e.g., breast cancer, prostate cancer, lung cancer, brain cancer, ovarian cancer, or any other cancer characterized by high Twist expression).

In another aspect, the invention provides a method for identifying a subject as having a breast carcinoma or ductal carcinoma in situ having high metastatic potential, the method involves detecting an increase in the level of Twist and CD44 polypeptides or nucleic acid molecules, and a reduction in the level of CD24 polypeptides or nucleic acid molecules in a biological sample from the subject relative to levels present in control, thereby identifying the breast carcinoma or ductal carcinoma in situ as having high metastatic potential.

In another aspect, the invention provides a method for characterizing a biological sample as lacking or having an undetectable level of estrogen receptor expression, the method involves detecting an increase in the level of Twist in the sample.

In another aspect, the invention provides a method of selecting a treatment for a subject identified as having breast cancer or ductal carcinoma in situ, the method involves detecting an increase in the level of Twist and CD44 polypeptides or nucleic acid molecules, and a reduction in the level of CD24 polypeptides or nucleic acid molecules in a biological sample from the subject relative to levels present in a control, where the levels are indicative of the therapy to be selected. In one embodiment, the presence of Twist⁺/CD44⁺/CD24^{-/low} cells is indicative that aggressive therapy should be selected. In another embodiment, the absence of Twist⁺/CD44⁺/CD24^{-/low} cells is indicative that less aggressive therapy should be selected.

In another aspect, the invention provides a method for treating a subject identified as having a breast cancer or ductal carcinoma in situ that contains Twist⁺/CD44⁺/CD24^{-/low} cells, the method involves administering to the subject a combination of Twist inhibitory nucleic acid molecule, a methylation inhibitor, and an HDAC inhibitor. In one embodiment, the Twist inhibitory nucleic acid molecule is an siRNA, shRNA, or antisense RNA. In another embodiment, the methylation inhibitor is any one or more of 5-azacytidine, 5-azadeoxycytidine, procainamide, zebularine, and RG108. In another embodiment, the HDAC inhibitor is any one or more of valproic acid, sodium butyrate, and Trichostatin A. In yet another embodiment, the method increases estrogen receptor expression.

In another aspect, the invention provides a method of monitoring a cancer therapy in a subject, the method involves detecting the level of Twist, CD44, and CD24 polypeptides or nucleic acid molecules in a biological sample from the subject relative to levels present in control obtained from the subject at an earlier time point, where a therapy that reduces or eliminates Twist and CD44 levels and/or increases CD24 levels is identified as effective. In one embodiment, the method reduces the number of Twist⁺/CD44⁺/CD24^{-/low} cells present in the biological sample. In one embodiment, the level is reduced by about 15% to about 70% (e.g., by 15, 20, 25, 30, 40, 50, 60, 70%).

In another aspect, the invention provides a method for isolating a breast cancer stem cell or a cell population enriched for such cells, the method involves selecting a cell that is Twist⁺/CD44⁺/CD24^{-/low}. In one embodiment, the cell is selected using FACS analysis. In another embodiment, the cell population contains at least about 15%, 25%, 50%, 75%, 85%, 95% or more Twist⁺/CD44⁺/CD24^{-/low} cells.

In another aspect, the invention provides an isolated Twist⁺/CD44⁺/CD24^{-/low} breast cancer stem cell. In one embodiment, the cell is capable of estrogen-independent growth and/or is tamoxifen-resistant.

In another aspect, the invention provides a cell population enriched for Twist⁺/CD44⁺/CD24^{-/low} breast cancer stem cells. In one embodiment, the cells are selected using FACS analysis. In another embodiment, the cell population contains at least about 15%, 25%, 50%, 75%, 85%, 95% or more Twist⁺/CD44⁺/CD24^{-/low} cells.

In another aspect, the invention provides a method of identifying an anti-cancer agent, the method involves contacting a Twist⁺/CD44⁺/CD24^{-/low} cell with an agent; and detecting a reduction in the survival, proliferation, or metastatic potential of the cell or an increase in cell death. In one embodiment, the method further involves measuring the level of Twist, CD44, and CD24 in the cell, where an agent that reduces or eliminates the level of Twist or CD44 or increases the level of CD24 in the cell is identified as an anti-cancer agent.

In another aspect, the invention provides a kit for the detection of a Twist⁺/CD44⁺/CD24^{-/low} cell, the kit contains reagents capable of specifically hybridizing or binding to Twist, CD44, and CD24 nucleic acid molecules or polypeptides.

In various embodiments of any of the above aspects or any other aspect of the invention delineated herein, the method further involves detecting one or more polypeptide or polynucleotide biomarkers that is any one or more of ABCC1, ABCG2, ALDH, CD10, CD133, choline kinase, CXCR4, lactate dehydrogenase, Lin negative, PROCR, ER, ESA, MUC1, and p27. In other embodiments of the above aspects, the biological sample is a liquid (e.g., a nipple aspirant, ductal lavage specimen, milk, urine, fecal matter) or tissue sample (e.g., a needle biopsy, or a tissue biopsy). In other embodiments of the above aspects, the sample is further characterized by grade. In other embodiments of the above aspects, the sample is further characterized for one or more of level of estrogen receptor expression, level of estrogen receptor promoter methylation, or level of estrogen receptor promoter histone deacetylation. In other embodiments of the above aspects, the method further involves detecting estrogen receptor (ER), a human epidermal growth factor receptor 2 (Her2), and/or a progesterone receptor (PR) in the sample. In still other embodiments, the polypeptide is detected in an immunoassay, an ELISA, immunocytochemistry, immunohistochemistry, flow cytometric analysis, radioimmunoassay, Western blot, and mass spectrometry, ¹H NMR. In other embodiments of the above aspects, the nucleic acid molecule is detected by a method selected from the group consisting of PCR, rtPCR, quantitative rtPCR, using a probe that hybridizes to the nucleic acid molecule, and microarray analysis.

In one aspect, the present invention provides methods and compositions for the identification of cancer stem cells. In a specific embodiment, the methods and compositions of the present invention may be used to identify breast cancer stem cells. In another embodiment, the methods and compositions of the present invention may be used to distinguish breast cancer stem cells within a breast tumor mass.

More specifically, the breast cancer stem cells may be identified using a set of biomarkers. In a particular embodiment, the biomarkers are cell surface markers. In another embodiment, the biomarkers may comprise Twist⁺/CD44⁺/CD24^{-/low}. In a further embodiment, the biomarker set Twist⁺/CD44⁺/CD24^{-/low} may be combined with at least one other breast cancer stem cell biomarker known to those of ordinary skill in the art including, but not limited to, Lin⁻, aldehyde dehyrdogenase 1 (ALDH⁺), CD10, CD133, CXCR4⁺, PROCR⁺, and ESA⁺. In a specific embodiment, the biomarker set comprises Twist⁺/CD44⁺/CD24^{-/low}/ALDH⁺.

In another embodiment, the biomarkers of the present invention may be used to identify other cancer stem cells including cells from such cancers as acute myeloid leukemia (AML), brain cancer, acute lymphoid leukemia (ALL), ovarian cancer, multiple myeloma, chronic myelogenous leukemia (CML), chronic lymphocytic leukemia (CLL), lymphoma, melanoma, ependymoma, prostate cancer, lung cancer, thyroid cancer, colorectal cancer, pancreatic cancer, bladder cancer, myelodysplastic syndrome (MDS), hairy cell leukemia, and stomach cancer.

In another aspect, the biomarkers of the present invention may be used to quantify the amount of breast cancer cells in a sample. Various methods known in the art can be used to detect and determine the amount of breast cancer stem cells in a sample.

In yet another aspect, the biomarkers of the present invention may be used to determine whether a cancer therapy is effective. In a specific embodiment, the biomarkers of the present invention (e.g., Twist⁺/CD44⁺/CD24^{-/low}) may be used to measure the amount of breast cancer stem cells in a patient sample at different time points before, during or after a cancer treatment regimen. The change in the amount of breast cancer cells in the samples over time indicates the effectiveness of the treatment regimen. In another embodiment, the amount of breast cancer stem cells may be measured in vivo using imaging techniques known to those of ordinary skill in the art. In a further embodiment, the present invention provides kits useful for identifying, quantifying, and/or monitoring breast cancer stem cells.

More specifically, in determining whether a cancer therapy is effective, the amount of breast cancer stem cells in a patient sample may be compared to the amount of breast cancer stem cells in a reference sample or to a predetermined reference range. A decrease or reduction in the amount of breast cancer stem cells in the patient sample (or patient) relative to the reference sample or the predetermined reference range indicates that the cancer treatment regimen is effective. An increase in the amount of breast cancer stem cells in the patient sample (or patient) relative to the reference sample or the predetermined reference range indicates that the cancer treatment regimen is ineffective. A reference sample may be a sample obtained from the patient from an earlier time (e.g., prior to undergoing the cancer treatment regimen), a sample obtained from a second patient having the same type of cancer that is in remission, or a sample obtained from a healthy person with no detectable cancer.

In an alternative embodiment, the present invention provides methods for determining the potential efficacy of a cancer therapy. For example, a sample from a breast cancer patient may be contacted in vitro with a potential anti-cancer therapeutic compound, and the amount of breast cancer stem cells in the contacted sample may be determined. A reduction in the amount of breast cancer stem cells in the contacted sample as compared to a reference sample, or to a predetermined range (including the untreated sample itself as a comparator control), indicates that the cancer therapy is efficacious for that cancer. The biomarkers of the present invention may further be used to target breast cancer stem cells for destruction. Antibodies specific to the biomarkers may target therapeutics to such breast cancer stem cells to induce apoptosis or differentiation with a loss of self-renewal capacity.

The present invention further provides methods for treating breast cancer. In one embodiment, the method comprises administering a cancer therapy to a breast cancer patient, and determining the amount of breast cancer stem cells prior to, during, and/or following therapy through the monitoring of breast cancer stem cells. In certain embodiments, the therapy may be continued, altered, or halted based on such monitoring. In another embodiment, the method comprises administering a cancer therapy to a breast cancer patient, and detecting a decrease in the amount of breast cancer stem cells through the monitoring of cancer stem cells, and continuing, altering, or halting therapy based on such monitoring.

The present invention also provides methods for enhancing the accuracy and reliability of histopathological scoring of tissue or biopsy preparations by pathologists.

Other features and advantages of the invention will be apparent from the detailed description, and from the claims.

### DEFINITIONS

By “Twist polypeptide” is meant a polypeptide or fragment thereof having at least about 85% amino acid identity to NCBI Accession No. NP_{—}000465.1, and having DNA binding activity.

By “Twist nucleic acid molecule” is meant a polynucleotide encoding a Twist polypeptide, NCBI Accession No. NM_{—}000474.3.

By “CD44 polypeptide” is meant a polypeptide or fragment thereof having at least about 85% amino acid identity to NCBI Accession No. NP_{—}000601.3 and having DNA binding activity.

By “CD44 nucleic acid molecule” is meant a polynucleotide encoding a CD44 polypeptide. An exemplary CD44 nucleic acid molecule is provided at NCBI Accession No. NM_{—}000610.3. Additional exemplary CD44 nucleic acid molecules include, but are not limited to, the following variant transcripts: variant 2, NCBI Accession No. NM_{—}001001389.1, Gene product: NP_{—}001001389.1; variant 3, NCBI Accession No NM_{—}001001390.1, Gene product: NP_{—}001001390.1; variant 4, NCBI Accession No. NM_{—}001001391.1, Gene product: NP_{—}001001391.1; variant 5, NCBI Accession No. NM_{—}001001392.1, Gene product: NP_{—}001001392.1; variant 6, NCBI Accession No. NM_{—}001202555.1, Gene product: NP_{—}001189484.1; variant 7, NCBI Accession No. NM_{—}001202556.1, Gene product: NP_{—}001189485.1; and variant 8, NCBI Accession No. NM_{—}001202557.1, Gene product: NP_{—}001189486.1.

By “CD24 polypeptide” is meant a polypeptide or fragment thereof having at least about 85% amino acid identity to NCBI Accession No. NP_{—}037362.1. and having DNA binding activity.

By “CD24 nucleic acid molecule” is meant a polynucleotide encoding a CD24 polypeptide. An exemplary CD24 nucleic acid molecule is provided at NCBI Accession No. NM_{—}013230.2.

By “biologic sample” is meant any tissue, cell, fluid, or other material derived from an organism.

By “biopsy” is meant a sample of tissue removed from a subject for the purpose of diagnosis.

By “cancer stem cell” is meant a cell that can undergo self-renewal as well as abnormal proliferation. For example, a cancer stem cell can give rise to one cancer stem cell (i.e. self-renewal) and one neoplastic cell. Functional features of cancer stem cells are that they are tumorigenic; they can give rise to additional neoplastic cells by self-renewal; and/or they can give rise to non-tumorigenic neoplastic cells. Without being bound to any particular theory, cancer stem cells contribute to the development of metastatic cancer.

By “clinical aggressiveness” is meant the severity of the cancer or neoplasia. Aggressive cancers are more likely to metastasize than less aggressive cancers. While conservative methods of treatment are appropriate for less aggressive cancers, more aggressive cancers require more aggressive therapeutic regimens.

By “high metastatic potential” is meant that a neoplastic or cancer cell has, or has a propensity to develop, the ability to penetrate and infiltrate surrounding normal tissues.

By “Lin negative (Lin⁻)” is meant a genotype that results in the loss of at least the following cell surface markers: CD2, CD3, CD10, CD16, CD18, CD31, CD64, and CD140b

By “neoplasia” is meant any disease that is caused by or results in inappropriately high levels of cell division, inappropriately low levels of apoptosis, or both. For example, cancer is an example of a neoplasia. Examples of cancers include, without limitation, breast cancer, prostate cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “reference” is meant a standard of comparison. For example, the ABCC1, ABCG2, ALDH, CD10, CD133, choline kinase, CXCR4, lactate dehydrogenase, Lin, PROCR, ER, ESA, MUC1, and p27 polynucleotide or polypeptide level present in a patient sample may be compared to the level of said polypeptide or polynucleotide present in a corresponding healthy cell, cell population, cell sub-population, or tissue or in a neoplastic cell or tissue that lacks a propensity to metastasize.

By “periodic” is meant at regular intervals. Periodic patient monitoring includes, for example, a schedule of tests that are administered daily, bi-weekly, bi-monthly, monthly, bi-annually, or annually.

By “severity of neoplasia” is meant the degree of pathology. The severity of a neoplasia increases, for example, as the stage or grade of the neoplasia increases.

By “marker profile” is meant a characterization of the expression or expression level of two or more polypeptides or polynucleotides.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include cancer, pneumonia, down syndrome, cystic fibrosis, hepatitis, smallpox, and Conn's syndrome.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 17, 18, 19, 20, 21, 22, 23 or 24 nucleotides in length and has a 1-2 base overhang at its 3′ end. These dsRNAs can be introduced to an individual cell or to a whole animal; for example, they may be introduced systemically via the bloodstream. Such siRNAs are used to down regulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A is a schematic diagram showing the pleiotropic effects of Twist in promoting breast cancer initiating cells (BCICs) and metastasis.

FIGS. 1B and 1C show immunoblots and two-dimensional dot plots, respectively. FIG. 1A depicts immunoblots showing the downregulation of Twist in MCF-10A/Twist, MCF7/Twist, and MDA-MB-231 breast cancer cell lines by short hairpin RNA against Twist delivered using lentiviral vectors (OpenBiosystems, Huntsville, Ala.). Lower panels show Twist transient expression levels in MCF10A and MCF-7 cell lines expressing Twist by retroviral delivery. The antibody against Twist was generated in-house and was validated (Mironchik et al. (2005) Cancer Res. 2005; 65:10801-9). Actin was scored as loading control. FIG. 1B depicts dot plots of flow cytometry analysis of various cell lines for CD44 and CD24 expression. Unstained and single-antibody stained cells were used as controls for setting the quadrants. The events in the lower right quadrant represent the CD44⁺/CD24^{-/low} subpopulation. The analyzed data are represented as dot plots of (first row) immortalized normal mammary epithelial cell line MCF-10A and breast cancer cell lines MCF-7 and MDA-MB-231, (second row) stable Twist overexpressing cell lines MCF-10A/Twist and MCF-7/Twist, (third row) transiently transduced Twist-expressing MCF-10A and MCF-7, and (fourth row) Twist knockdown in MCF-10A/Twist, MCF-7/Twist, and MDA-MB-231 breast cancer cells. Ten thousand viable cells were gated for each dot plot acquisition. Results are representative of five separate experiments.

FIGS. 2A and 2B show immunoblots of epithelial-mesenchymal marker expression in Twist cells. FIG. 2A shows immunoblots of the epithelial marker E-cadherin and the mesenchymal marker vimentin in transient knockdown of Twist in stable Twist-expressing cell lines MCF-10A/Twist and MCF-7/Twist. FIG. 2B shows immunoblots of E-cadherin and vimentin in MCF-7 and MCF-7/Twist cells transiently expressing Twist. Transduced cells were lysed and immunoblotted with antibodies against E-cadherin, vimentin, and Twist (Mironchik et al. (2005) Cancer Res. 2005; 65:10801-9). Actin was used as loading control.

FIGS. 3A, 3B-3C, and 3D show photomicrographs, histograms, and flow cytometric histograms, respectively, describing efflux studies in MCF-7 and MCF-7/Twist cells. FIG. 3A shows representative photomicrographs of Hoechst efflux staining in MCF-7/Twist cells compared with parental MCF-7 cells. The cells, after efflux, were photographed using a Nikon Eclipse 80i fluorescence microscope. FIG. 3B is a histogram showing quantification of fluorescence intensity per cell. Cell fluorescence (n=6) in the blue channel was analyzed using dedicated software developed in IDL programming environment that provides an operator-free segmentation of images and determines the average relative fluorescence per cell. Three images were analyzed per sample. FIG. 3C is a histogram showing expression of drug transporters ABCG2, ABCC1, and ABCA1 in MCF7/Twist and parental MCF-7 cells. FIG. 3D presents histograms showing efflux of Rhodamine 123 stain in MCF-7 (left graph) and in MCF-7/Twist (right graph) cells. The amount of Rhodamine 123 dye in the cells was determined by flow cytometry. Results are representative of three separate experiments.

FIG. 4 shows dot plots of various cell lines analyzed by flow cytometry for ALDH activity in Twist-dysregulated breast cells. Cells were treated with ALDEFLUOR in the presence or absence of ALDH inhibitor DEAB. After treatment, the samples were analyzed by flow cytometry for the presence of ALDH bright cells. The ALDH bright region was based on the control DEAB sample that was gated to have less than five events. The cell percentage numbers for ALDH positivity are indicated in the bottom right of each histogram. The values presented are the averages of three independent experiments. The analyzed data are represented as dot plots of (first row) parental MCF-10A and MCF-7 breast cancer cell lines, (second row) stable Twist-overexpressing MCF-10A/Twist and MCF-7/Twist breast cancer cell lines, (third row) breast cell lines MCF-10A and MCF-7 transiently transduced with Twist-expressing retroviral constructs, and (fourth row) Twist knockdown breast cancer cell lines MCF-10A/Twist and MCF-7/Twist. Results are representative of five separate experiments.

FIGS. 5A, 5B, and 5C-5D show dot plots, histograms, and photomicrographs, respectively, illustrating self-renewal and mammosphere formation of Twist-expressing MCF7 cells. MCF-7/Twist cells were stained with CD44 and CD24 antibodies and flow sorted to purify the CD44⁺/CD24^{-/low} subpopulation. The purified MCF-7/Twist CD44⁺/CD24^{-/low} subpopulation was propagated in culture, and the percentage of CD44 and CD24 cells was estimated by flow cytometry. FIG. 5A shows dot plots of chronological changes in the CD44⁺/CD24^{-/low} low subpopulation during a period of 13 generations. FIG. 5B shows histograms of qRT-PCR analysis of Twist, CD44, and CD24 transcript levels in the purified CD44⁺/CD24^{-/low} versus CD44⁺/CD24⁺ subpopulations of cells. FIGS. 5C and 5D show mammosphere formation by stable Twist overexpressing cell lines, MCF-7/Twist, and MCF-10A/Twist. Top rows show phase-contrast photomicrographs of mammospheres at a magnification of 20×. The lower rows show viability of the cells as indicated by the fluorescence of Calcein-AM stain. Data are represented as histograms to the right of each set of four photomicrographs. Experiments were performed in quadruplicates, and an average of five fields per well were counted.

FIGS. 6A and 6B show a schematic diagram and a histogram, respectively, which together demonstrate transcriptional regulation of CD24 expression by Twist. FIG. 6A is a schematic representation of the CD24 promoter sequence showing the location of putative TWIST binding sites relative to the transcription start site (+1). Tick marks denote the canonical E-box sequences (CANNTG) to which Twist can potentially bind. The numbers above the tick marks indicate the relative position of E-box sequences within the promoter region. FIG. 6B shows the results of promoter assays represented as a histogram showing normalized luciferase readings in CD24 promoter activity. Reporter assays were performed using CD24 promoter-reporter constructs in MCF-7 with exogenous Twist expression plasmid added and estimated for 2 days.

FIG. 7 shows an immunoblot analysis of cell extracts from MCF-7 and MCF-7/Twist cells scored for CD24 and Twist protein expression. Actin was used as a loading control.

FIGS. 8A and 8B show a gel and a histogram, respectively, that demonstrate transcriptional regulation of CD24 expression by Twist. FIG. 8A shows in vivo binding of Twist protein to the CD24 promoter sequence, while FIG. 8B is a histogram quantifying the results of FIG. 8B. ChIP was carried out using MCF-7/Twist cells and analyzed using CD24 promoter-specific primers by PCR. Identical volumes from the final precipitate were used for the PCRs except for the input, which was diluted 10-fold.

FIG. 9 shows immunohistochemical determination of Twist expression in normal mammary epithelium versus primary breast carcinomas as seen in representative photomicrographs of sections of breast tissue stained with a Twist-specific rabbit polyclonal antibody. Sections of normal epithelium show little or no staining, while breast tissue samples from ductal carcinoma in situ and invasive ductal carcinoma present a dark staining pattern.

FIG. 10 depicts a graph that depict growth of low inoculums of purified CD44⁺/CD24^{-/low} and CD44⁺/CD24⁺ sub-populations in SCID mice. FIG. 10 is a graphical representation of growth rates of orthotopic xenograft tumors using flow sorted cells from the Twist⁺/CD44⁺/CD24^{-/low} and CD44⁺/CD24⁺ sub-populations. Four mice per inoculum were used for this study, and significance was analyzed by two sided t-test (**p<0.05, ***p<0.005).

FIG. 11 shows photomicrographs that depict growth of low inoculums of purified CD44⁺/CD24^{-/low} and CD44⁺/CD24⁺ sub-populations in SCID mice. FIG. 11 shows Twist immunohistochemical staining of representative tumors from the CD44⁺/CD24^{-/low} and CD44⁺/CD24⁺ sub-populations. This is a repeat of FIGS. 7 and 8.

FIGS. 12A and 12B are a histogram and immunoblot, respectively, that assess MUC1 expression in breast cancer cell lines. FIG. 12A depicts qRT-PCR performed on RNA extracted from the different breast cancer cell lines listed on the X-axis. The primers used spanned the region between +2425 and +2666 with respect to the transcription start site. The experiments were done twice (experimental duplicate) as well as in technical triplicates. The relative expression shown is normalized to that of MCF7. FIG. 12B shows an immunoblot analysis of MUC1 expression in breast cancer cell lines using the HMFG-2 antibody. This antibody recognizes the hypoglycosylated form of MUC1 protein. The molecular weight shown is approximately 200 kDa.

FIG. 13 displays histograms indicating MUC1 expression in MCF7/Twist and MCF 10A/Twist cells. Cells were harvested and stained with FITC conjugated MUC1 antibody. A parallel control tube with the same number of cells but without antibody was used as a staining control. Data was acquired and analyzed on a FACScan instrument. The boxed numbers indicate the normalized geometric means of signal intensities, where lower intensities represent lower expression levels.

FIGS. 14A-14C depicts a schematic p27 promoter representation, a bar graph, and an immunoblot, respectively, that together indicate that Twist down-regulates p27 levels in breast cancer cells. FIG. 14A is a schematic representation of the p27 promoter sequence showing the location of the putative TWIST binding sites relative to the translation start site (+1) as asterisks that denote the canonical E-box sequence (CANNTG) to which Twist may directly bind. FIG. 14B shows the results of a transient transfection assay using p27 promoter-reporter constructs in MCF-7 (exogenous Twist expression plasmid added) and in MCF-7/Twist cells. FIG. 14C is a Western blot analyses of cell extracts from MCF-7 and MCF-7/Twist cells respectively. Antibodies used were against Twist and p27 (Santa Cruz) and β-actin.

FIGS. 15A and 15B are gels depicting the results of chromatin immunoprecipitation (ChIP) experiments to assess in vivo binding of Twist protein to the p27 promoter sequence. ChIP was carried out following established protocols using MCF-7/Twist and Hs578 T cells and analyzed using p27 promoter specific primers by polymerase chain reaction (PCR). Primers amplified a 241 base pair fragment. FIGS. 15A (MCF-7/Twist) and 15B (Hs578T) show gels in which lane 1 is a DNA ladder, lane 2 is total input chromatin (diluted 10 times), lane 3 is an anti-acetyl-Histone H3 precipitation, lane 4 is a non-specific antibody precipitation control, lane 5 is a no antibody control, lane 6 is a no chromatin-Twist specific antibody control, and lane 7 is an anti-Twist antibody precipitation. Identical volumes from the final precipitate were used for the PCR reactions. Molecular weights indicated to the left of the gels are given in base pairs.

FIG. 16 shows an orthotopic xenograft mouse model of metastatic breast cancer with H&E staining of representative sections of lungs from three different mice showing metastatic areas (black arrows within the tumor section).

FIG. 17 depicts an experimental model of breast cancer metastasis. MCF7/Twist cells were injected into the left ventricle and bone metastasis was identified using a Faxitron instrument. Top panels show the X-ray pictures of the tibia following two months of incubation, while the bottom panels show the H & E section of the representative tibias. The yellow circle in the top right panel depicts bone destruction, and the black arrow in the bottom right panel indicates MCF7/Twist cells within the bone matrix.

FIGS. 18A, 18B-18C, and 18D show an immunoblot, ¹H MR spectroscopy profiles, and a bar graph, respectively. FIG. 18A is an immunoblot showing increased choline kinase expression in MCF-7 Twist cells. FIGS. 18B (MCF7 wild type cells) and 18C (MCF-7 Twist over-expressing cells) show ¹H MR spectroscopy data in which spectral regions from the glycerophosphocholine (GPC) and phosphocholine (PC) regions are displayed. Spectra were obtained from comparable numbers of cells in each experiment. FIG. 18D shows PC levels in MCF-7 and MCF-7/Twist cells.

FIGS. 19A and 19B show lactate and [H⁺] ion levels, respectively, in the medium of MCF-7/Twist and MCF-7 cells. Following growth of MCF-7/Twist and MCF-7 cells, media from two independent wells were used to estimate lactate (FIG. 19A) and pHe (FIG. 19B) levels on an ABL 700 blood gas analyzer. pHe was converted to the moles of [H⁺] using the formula [H⁺]=10 (−pH). Lactate and [H⁺] ion levels were normalized to cell counts.

FIG. 20 shows cross-sectional gradient-echo images obtained with a saturation recovery time of 1 s from mouse with lung metastasis (top panels, red boxes). The lower panel shows a magnification of the regions outlined in the red boxes in the top panel images. Uptake of the contrast agent is evident by the signal enhancement in the nodules. Slice thickness 1 mm, inplane resolution 0.2 mm.

FIG. 21 shows the histopathology of H & E stained lung sections obtained from the same mouse imaged in FIG. 20. The arrows indicate the foci of some of the lung metastatic nodules.

FIG. 22 shows representative optical images of primary and metastatic breast tumors. Images show the fluorescence of two mice at 8 weeks post MDA-MB-231-tdTomato cell injection. The arrowhead indicates contra-lateral breast metastasis.

FIGS. 23A, 23B, 23C-23D, and 23E show an immunoblot, a histogram, schematic diagrams, and a gel, respectively, that assess Twist regulation of ER by direct promoter binding. FIG. 23A shows total proteins from three tumorigenic (MCF-7, T-47D, and Hs578T) and two metastatic (MDA-MB-231 and MCF-7/Twist) breast cancer cell lines immunoblotted for Twist, ER, and β-actin. FIG. 23B shows a histogram depicting relative expression of Twist and ER mRNA from cell lines analyzed by qRT-PCR using Twist specific (5′-GGACAAGCTGAGC AAGATTCAGA-3′ and 5′-TCTGGAGGACCTGGTAGAGGAA-3′) and ER specific (5′-GA CAGGGAGCTGGTTCACAT-3′ and 5′-AGGATCTCTAGCCAGGCACA-3′) primer sets. Experiments were repeated three times in duplicates. Error bars depict S.D. FIG. 23C shows a schematic representation of ER promoter constructs showing the location of putative Twist binding E-boxes, denoted by short vertical lines. ChIP primers are denoted by arrows. Promoter reporter assay results are displayed in the histogram on the right. Experiments were repeated five times in duplicates. Error bars depict S.D. FIG. 23D shows a schematic displaying Twist wild-type (wt) and mutant constructs. Stop codons and the DNA binding basic domain (B) and the helix-loop-helix (HLH) domain are indicated. Promoter activity is displayed by the histogram on the right. All luciferase activities were normalized to the activity of wild type Twist. Experiments were repeated twice in duplicates. Error bars depict S.E.M. FIG. 23E shows the results of ChIP experiments carried out using MCF-7/Twist and Hs578T cells, and analyzed using ER promoter specific primers in PCR. Lane M is a molecular weight marker, lane 1 is a total input chromatin, lane 2 is acetyl histone H3 precipitation, lane 3 is a non-specific antibody precipitation, lane 4 is a no antibody control, lane 5 is a no chromatin with Twist antibody control, lane 6 is a Twist antibody ChIP. Identical volumes from the final precipitate were used for PCR reactions (except total input chromatin sample, which was diluted 100×).

FIGS. 24A and 24B show histograms depicting cell cycle profiles of MCF-7 and MCF-7/Twist cells based on the results of the flow cytometric acquisition and cell cycle analysis of MCF-7 and MCF-7/Twist cells following hormone treatment. Cell cycle stage values were calculated by the Dean-Jett-Fox model. Experiments were independently repeated four times.

FIGS. 25A, 25B, 25C-25D, and 25E-25F show line charts, a scatter plot, line charts, and xenograft transverse slices and corresponding histograms, respectively. FIG. 25A is a line chart showing growth of 1×10⁶ MCF-7 cells (without E2, triangles; with E2, circles) and MCF-7/Twist cells (without E2, squares) orthotopically implanted into female SCID mice and allowed to grow for the time indicated. Tumors were measured weekly. FIG. 25B is a scatterplot depicting relative differences in ER and Twist transcript levels in MCF-7/Twist tumors from mice (n=4) as determined by qRT-PCR. FIGS. 25C and 25D are line charts showing growth of MCF-7/Twist and MCF-7 tumors over 8 weeks treated with tamoxifen. FIGS. 25E and 25F are representative false color coded MRI generated 3-D transverse slices of MCF-7 and MCF-7/Twist xenografts in the mammary fat pad. Red and green represent the distributions of vascular volume (VV) and vascular permeability surface area product (PS), respectively. Averaged values from all mice are written in the figures as well as displayed on the histograms at the right. Gray-scale images represent the mouse body; while tumors are seen on top and indicated by “T.” Images depicted are a representative sample of five mice for MCF-7(+E2), six mice for MCF-7/Twist(−E2), and six mice for MCF-7/Twist(+E2).

FIGS. 26A, 26B, and 26D-26G show histograms and FIGS. 26C and 26H-26I show immunoblots, all of which show the effects on promoter methylation of Twist knock down and over-expression. FIGS. 26A and 26B are histograms depicting changes in Twist and ER RNA expression after Twist and shTwist mediated knock up and knock down in MCF-7 and MCF-7/Twist cells respectively. Transcript levels were estimated by qRT-PCR and derived from three independent experiments in duplicates. Error bars depict S.D. FIG. 26C depicts immunoblots of Twist knock up and knock down cell lines scored for Twist and ER. FIG. 26D is a histogram depicting changes in relative binding of ER to an ERE luciferase plasmid in MCF-7, MCF-7/Twist, and shTwist mediated Twist knock down MCF-7/Twist cells. Experiments were repeated three times in duplicates. Error bars depict S.D. FIG. 26E is a histogram depicting basal ER promoter methylation levels of the cell lines. Experiments were repeated twice in duplicates. Error bars depict S.D. FIG. 26F is a histogram displaying ER promoter methylation in cell lines after Twist over-expression and knock down. Experiments were repeated twice in duplicates. Error bars depict S.D. FIG. 26G is a histogram depicting the change in ER expression in MCF-7/Twist cells treated with 1 μM AZA and 10 μM VPA and assayed by qRT-PCR. Experiments were repeated twice in duplicates. Error bars depict S.D. FIG. 26H shows immunoblots of ER re-expression in MCF-7/Twist cells after treatment by AZA, VPA, and AZA+VPA in combination. FIG. 26I shows immunoblots of co-immunoprecipitation of MCF-7/Twist lysates. Twist antibodies were used for the co-immunoprecipitation, and immunoblots were probed with HDAC1 and DNMT3B.

FIG. 27 is a histogram that shows the correlation between Twist and ER mRNA levels in various grades of human breast tumors, including: grade I (n=6), grade II (n=12), and grade III (n=13). Spearman's rank correlation test was performed on the samples and showed an inverse correlation. Four normal samples were used to normalize expression. Error bars display S.E.M.

FIG. 28 shows a model for the regulation of ER by Twist in which Twist up-regulation causes the down-regulation of ER by direct transcriptional action.

### DETAILED DESCRIPTION OF THE INVENTION

The invention features compositions and methods that are useful for the diagnosis, treatment and prevention of breast cancer, as well as for characterizing the breast cancer to determine a subject's prognosis and aid in treatment selection.

The invention further provides compositions and methods for monitoring a patient identified as having breast cancer. The present invention is based, at least in part, on the following discoveries: first, that Twist⁺/CD44⁺/CD24^{-/low} are useful to identify cancer stem cells; and second, that increased Twist expression in a breast cancer sample indicates that the breast cancer is Estrogen-receptor negative. The identification of Twist⁺/CD44⁺/CD24^{-/low} breast cancer stem cells in a subject sample identifies the subject as having an aggressive form of breast cancer with a high metastatic potential. Importantly, Twist expression can serve as a surrogate marker for Estrogen receptor expression (i.e. lack of expression). Conventional diagnostic methods for characterizing estrogen receptor are not quantifiable, notoriously unreliable and lack reproducibility.

Accordingly, the invention provides improved diagnostic compositions that are useful for identifying subjects as having, or having a propensity to develop, metastatic breast cancer. The invention further provides compositions and methods for identifying a biological sample as lacking Estrogen receptor by characterizing the presence of Twist polypeptide or nucleic acid molecule expression. The invention further provides methods of using these compositions to identify a subject's prognosis, select a treatment regimen, and monitor the subject before, during or after treatment.

### Diagnostics

The present invention features diagnostic assays for the detection of a biomarker set that is correlated with breast cancer stem cells with high metastatic potential. In one embodiment, levels of Twist, CD44, and CD24 are measured in a subject sample to identify the presence of breast cancer stem cells and/or to characterize the breast cancer's metastatic potential. If desired, the sample is further characterized by detecting one or more of the following markers ABCC1, ABCG2, ALDH, CD10, CD133, choline kinase, CXCR4, lactate dehydrogenase, Lin⁻, PROCR, ER, ESA, MUC1, and p27. In other embodiments, the sample is further characterized by detecting estrogen receptor(s) (ER), a human epidermal growth factor receptor 2 (Her2), and/or a progesterone receptor(s) (PR). Samples that lack or have undetectable levels of estrogen receptor(s) (ER), a human epidermal growth factor receptor 2 (Her2), and/or a progesterone receptor(s) (PR) are termed “triple negative.”

Standard methods may be used to measure levels of a biomarker in any biological sample. Biological samples include tissue samples (e.g., cell samples, biopsy samples) and bodily fluids, including, but not limited to, nipple aspirant, ductal lavage, milk, blood, blood serum, and plasma. Other biological samples useful in methods for identifying an epithelial carcinoma (e.g., breast cancer, prostate cancer, colon, lung cancer, brain cancer, ovarian cancer, or any other cancer characterized by high Twist expression). Methods for measuring levels of polypeptides include immunoassay, ELISA, western blotting and radioimmunoassay. Such assays may use an anti-Twist antibody as described in Mironchik et al. (2005) Cancer Res. 2005; 65:10801-9. Elevated levels of Twist and CD44 in combination with reduced levels of CD24 (i.e. Twist⁺/CD44⁺/CD24^{-/low}) are considered a positive indicator of breast cancer stem cells having high metastatic potential. The increase in Twist and CD44 levels may be by at least about 10%, 25%, 50%, 75% or more. The decrease in CD24 levels may be by at least about 10%, 25%, 50%, 75% or more. In one embodiment, the Twist⁺/CD44⁺/CD24^{-/low} biomarker profile is indicative of breast cancer with high metastatic potential. In another embodiment, levels of the biomarkers Twist, CD44, and CD24 are used to distinguish a benign breast lesion from breast cancer or from metastatic breast cancer. In another embodiment, levels of Twist, CD44, and CD24, in combination with levels of any one or more of the following biomarkers ABCC1, ABCG2, ALDH, CD10, CD133, choline kinase, CXCR4, lactate dehydrogenase, Lin, PROCR, ER, ESA, MUC1, and p27 are used to characterize the breast cancer.

Any suitable method can be used to detect one or more of the markers described herein. Successful practice of the invention can be achieved with one or a combination of methods that can detect and, preferably, quantify the markers. These methods include, without limitation, hybridization-based methods, including those employed in biochip arrays, mass spectrometry (e.g., laser desorption/ionization mass spectrometry), fluorescence (e.g. sandwich immunoassay), surface plasmon resonance, ellipsometry and atomic force microscopy. Expression levels of markers (e.g., polynucleotides or polypeptides) are compared by procedures well known in the art, such as RT-PCR, Northern blotting, Western blotting, flow cytometry, immunohistochemistry, binding to magnetic and/or antibody-coated beads, in situ hybridization, fluorescence in situ hybridization (FISH), flow chamber adhesion assay, ELISA, microarray analysis, or colorimetric assays. Methods may further include, one or more of electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n, quadrupole mass spectrometry, fourier transform mass spectrometry (FTMS), and ion trap mass spectrometry, where n is an integer greater than zero.

Detection methods may include use of a biochip array. Biochip arrays useful in the invention include protein and polynucleotide arrays. One or more markers are captured on the biochip array and subjected to analysis to detect the level of the markers in a sample.

Markers may be captured with capture reagents immobilized to a solid support, such as a biochip, a multiwell microtiter plate, a resin, or a nitrocellulose membrane that is subsequently probed for the presence or level of a marker. Capture can be on a chromatographic surface or a biospecific surface. For example, a sample containing the markers, such as serum, may be used to contact the active surface of a biochip for a sufficient time to allow binding. Unbound molecules are washed from the surface using a suitable eluant, such as phosphate buffered saline. In general, the more stringent the eluant, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. In one embodiment, mass spectrometry, and in particular, SELDI, is used. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

Mass spectrometry (MS) is a well-known tool for analyzing chemical compounds. Thus, in one embodiment, the methods of the present invention comprise performing quantitative MS to measure the serum peptide marker. The method may be performed in an automated (Villanueva, et al., Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos: 20050023454; 20050035286; U.S. Pat. No. 5,800,979 and references disclosed therein.

The protein fragments, whether they are peptides derived from the main chain of the protein or are residues of a side-chain, are collected on the collection layer. They may then be analyzed by a spectroscopic method based on matrix-assisted laser desorption/ionization (MALDI) or electrospray ionization (ESI). The preferred procedure is MALDI with time of flight (TOF) analysis, known as MALDI-TOF MS. This involves forming a matrix on the membrane, e.g. as described in the literature, with an agent which absorbs the incident light strongly at the particular wavelength employed. The sample is excited by UV, or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are commercially available from PerSeptive Biosystems, Inc. (Framingham, Mass., USA) and are described in the literature, e.g. M. Kussmann and P. Roepstorff, cited above.

Magnetic-based serum processing can be combined with traditional MALDI-TOF. Through this approach, improved peptide capture is achieved prior to matrix mixture and deposition of the sample on MALDI target plates. Accordingly, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on the collecting membrane, and the array may be analyzed. Subsequently, automated output of the results is provided by using the ExPASy server, as at present used for MIDI-TOF MS and to generate the data in a form suitable for computers.

Other techniques for improving the mass accuracy and sensitivity of the MALDI-TOF MS can be used to analyze the fragments of protein obtained on the collection membrane. These include the use of delayed ion extraction, energy reflectors and ion-trap modules. In addition, post source decay and MS-MS analysis are useful to provide further structural analysis. With ESI, the sample is in the liquid phase and the analysis can be by ion-trap, TOF, single quadrupole or multi-quadrupole mass spectrometers. The use of such devices (other than a single quadrupole) allows MS-MS or MSn analysis to be performed. Tandem mass spectrometry allows multiple reactions to be monitored at the same time.

Capillary infusion may be employed to introduce the marker to a desired MS implementation, for instance, because it can efficiently introduce small quantities of a sample into a mass spectrometer without destroying the vacuum. Capillary columns are routinely used to interface the ionization source of a MS with other separation techniques including gas chromatography (GC) and liquid chromatography (LC). GC and LC can serve to separate a solution into its different components prior to mass analysis. Such techniques are readily combined with MS, for instance. One variation of the technique is that high performance liquid chromatography (HPLC) can now be directly coupled to mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem MS experiments. FTMS is based on the principle of a charged particle orbiting in the presence of a magnetic field. Coupled to ESI and MALDI, FTMS offers high accuracy with errors as low as 0.001%.

In one embodiment, the marker qualification methods of the invention may further comprise identifying significant peaks from combined spectra. The methods may also further comprise searching for outlier spectra. In another embodiment, the method of the invention further comprises determining distant dependent K-nearest neighbors.

In another embodiment of the method of the invention, an ion mobility spectrometer can be used to detect and characterize serum peptide markers. The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify a marker or other substances in a sample. One advantage of ion mobility spectrometry is that it can operate at atmospheric pressure.

In an additional embodiment of the methods of the present invention, multiple markers are measured. The use of multiple markers increases the predictive value of the test and provides greater utility in diagnosis, toxicology, patient stratification and patient monitoring. The process called “Pattern recognition” detects the patterns formed by multiple markers greatly improves the sensitivity and specificity of clinical proteomics for predictive medicine. Subtle variations in data from clinical samples indicate that certain patterns of protein expression can predict phenotypes such as the presence or absence of a certain disease, a particular stage of cancer progression, or a positive or adverse response to drug treatments.

Expression levels of particular nucleic acids or polypeptides (e.g., Twist, CD44, CD24, ABCC1, ABCG2, ALDH, CD10, CD133, choline kinase, CXCR4, lactate dehydrogenase, Lin, PROCR, ER, ESA, MUC1, and p27) are correlated with breast cancer having a propensity to metastasize, and thus are useful in diagnosis. Antibodies that bind a polypeptide described herein, as well as oligonucleotides or longer fragments that hybridize to a nucleic acid sequence encoding one or more of Twist, CD44, CD24, ABCC1, ABCG2, ALDH, CD10, CD133, choline kinase, CXCR4, lactate dehydrogenase, Lin⁻, PROCR, ER, ESA, MUC1, and p27 polypeptides, or any other method known in the art may be used to monitor expression of a polynucleotide or polypeptide of interest.

Detection of an alteration relative to the level present in a normal, reference sample (e.g., a normal breast cancer sample, or a non-metastatic breast cancer sample) can be used as a diagnostic indicator of the presence of breast cancer stem cells with high metastatic potential. In particular embodiments, the expression profiles of Twist, CD44, and CD24, in combination with the expression profile of any one or more of the following biomarkers ABCC1, ABCG2, ALDH, CD10, CD133, choline kinase, CXCR4, lactate dehydrogenase, Lin⁻, PROCR, ER, ESA, MUC1, and p27 is indicative of breast cancer or the propensity to develop metastatic breast cancer. In other embodiments, a 2, 3, 4, 5, 6, 7, 8, 9, 10, 11, 12, 13, 14, 15, 16, 17, 18, 19, 20, 21, 22, 23, 24, or 25-fold change (increase or decrease) in the level of a marker of the invention is indicative of breast cancer stem cells. In yet another embodiment, an expression profile that characterizes alterations in the expression of three or more markers is correlated with a particular disease state (e.g., breast cancer with high metastatic potential). In one embodiment, a breast cancer can be monitored using the methods and compositions of the invention.

In one embodiment, the level of one or more markers is measured on at least two different occasions and an alteration in the levels as compared to normal reference levels over time is used as an indicator of breast cancer or the propensity to develop metastatic breast cancer. The level of marker in the bodily fluids (e.g., nipple aspirant, ductal lavage specimen, milk, blood, blood serum, plasma) of a subject having breast cancer or the propensity to develop such a condition may be altered by as little as 10%, 20%, 30%, or 40%, or by as much as 50%, 60%, 70%, 80%, or 90% or more relative to the level of such marker in a normal control. In general, levels of Twist, CD44, and CD24 are present at low levels in a healthy subject (i.e., those who do not have and/or who will not develop breast cancer). The diagnostic methods described herein can be used individually or in combination with any other diagnostic method described herein for a more accurate diagnosis of the presence or severity of breast cancer.

The diagnostic methods described herein can also be used to monitor and manage breast cancer, or to reliably distinguish breast cancer from metastatic breast cancer.

As indicated above, the invention provides methods for aiding a human cancer diagnosis using three or more markers, as specified herein. These markers can be used alone, in combination with other markers in any set, or with entirely different markers in aiding human cancer diagnosis. The markers are differentially present in samples of a human cancer patient and a normal subject in whom human cancer is undetectable. Therefore, detection of three or more of these markers in a person would provide useful information regarding the probability that the person may have breast cancer or regarding the aggressiveness or metastatic potential of the cancer.

The detection of the peptide markers is then correlated with a probable diagnosis of breast cancer or the propensity to develop metastic disease. The measurement of markers may also involve quantifying the markers to correlate the detection of markers with a probable diagnosis of cancer. Thus, if the amount of the markers detected in a subject being tested is different compared to a control amount (i.e., higher or lower than the control), then the subject being tested has a higher probability of having cancer, or a more aggressive form of cancer.

The correlation may take into account the amount of the marker or markers in the sample compared to a control amount of the marker or markers (e.g., in normal subjects or in non-cancer subjects such as where cancer is undetectable). A control can be, e.g., the average or median amount of marker present in comparable samples of normal subjects in normal subjects or in non-cancer subjects, such as where cancer is undetectable. The control amount is measured under the same or substantially similar experimental conditions as in measuring the test amount. As a result, the control can be employed as a reference standard, where the normal (non-cancer) phenotype is known, and each result can be compared to that standard, rather than re-running a control.

Accordingly, a biomarker profile may be obtained from a subject sample and compared to a reference marker profile obtained from a reference population, so that it is possible to classify the subject as belonging to or not belonging to the reference population. The correlation may take into account the presence or absence of the markers in a test sample and the frequency of detection of the same markers in a control. The correlation may take into account both of such factors to facilitate determination of cancer status.

In certain embodiments of the methods of qualifying cancer status, the methods further comprise managing subject treatment based on the status. The invention also provides for such methods where the markers (or specific combination of markers) are measured again after subject management. In these cases, the methods are used to monitor the status of the cancer, e.g., response to cancer treatment, remission of the disease or progression of the disease.

Any marker, individually, is useful in aiding in characterizing breast cancer. First, the selected marker is detected in a subject sample using the methods described herein. Then, the result is compared with a control that distinguishes a sample that comprises breast cancer stem cells. As is well understood in the art, the techniques can be adjusted to increase sensitivity or specificity of the diagnostic assay depending on the preference of the diagnostician.

While individual markers are useful diagnostic markers, in some instances, a combination of markers provides greater predictive value than single markers alone. The detection of a plurality of markers (or absence thereof, as the case may be) in a sample can increase the percentage of true positive and true negative diagnoses and decrease the percentage of false positive or false negative diagnoses. Thus, preferred methods of the present invention comprise the measurement of more than one marker (e.g. Twist, CD44, and CD24).

### Microarrays

As reported herein, a number of markers (e.g., Twist, CD44, CD24, ABCC1, ABCG2, ALDH, CD10, CD133, choline kinase, CXCR4, lactate dehydrogenase, Lin, PROCR, ER, ESA, MUC1, and p27) have been identified that are associated with breast cancer stem cells. Methods for assaying the expression of these polypeptides and the polynucleotides encoding them are useful for characterizing breast cancer. In particular, the invention provides diagnostic methods and compositions useful for identifying a polypeptide expression profile that identifies a subject as having or having a propensity to develop breast cancer or metastatic breast cancer. Such assays can be used to measure an alteration in the level of a polypeptide.

The polypeptides and nucleic acid molecules of the invention are useful as hybridizable array elements in a microarray. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Protein Microarrays

Proteins (e.g., Twist, CD44, CD24, ABCC1, ABCG2, ALDH, CD10, CD133, choline kinase, CXCR4, lactate dehydrogenase, Lin⁻, PROCR, ER, ESA, MUC1, and p27) may be analyzed using protein microarrays. Such arrays are useful in high-throughput low-cost screens to identify alterations in the expression or post-translation modification of a polypeptide of the invention, or a fragment thereof. In particular, such microarrays are useful to identify a protein whose expression is altered in breast cancer. In one embodiment, a protein microarray of the invention binds a marker present in a subject sample and detects an alteration in the level of the marker. Typically, a protein microarray features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

The protein microarray is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as nipple aspirant, ductal lavage, milk, blood, blood serum, plasma); a homogenized tissue sample (e.g. a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

### Nucleic Acid Microarrays

To produce a nucleic acid microarray, oligonucleotides may be synthesized or bound to the surface of a substrate using a chemical coupling procedure and an ink jet application apparatus, as described in PCT application W095/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, preferably as a bodily fluid (e.g., nipple aspirant, ductal lavage specimen, milk) or tissue sample (e.g. a tissue sample obtained by biopsy). For some applications, cultured cells or other tissue preparations may be used. The mRNA is isolated according to standard methods, and cDNA is produced and used as a template to make complementary RNA suitable for hybridization. Such methods are known in the art. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct nucleic acid sequences simultaneously (e.g., Heller et al., Proc. Natl. Acad. Sci. 94:2150-2155, 1997). Preferably, a scanner is used to determine the levels and patterns of fluorescence.

### Diagnostic Kits

The invention provides kits for diagnosing or monitoring breast cancer, including metastatic disease, or for selecting a treatment for breast cancer. In one embodiment, the kit includes a composition containing at least one agent that binds a polypeptide or polynucleotide whose expression is altered in breast cancer (e.g., Twist, CD44, CD24, ABCC1, ABCG2, ALDH, CD10, CD133, choline kinase, CXCR4, lactate dehydrogenase, Lin, PROCR, ER, ESA, MUC1, and p27). In another embodiment, the invention provides a kit that contains an agent that binds a nucleic acid molecule whose expression is altered in breast cancer. In some embodiments, the kit comprises a sterile container which contains the binding agent; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, the kit is provided together with instructions for using the kit to characterize the breast cancer. The instructions will generally include information about the use of the composition for diagnosing a subject as having breast cancer or having a propensity to develop metastatic breast cancer. In other embodiments, the instructions include at least one of the following: description of the binding agent; warnings; indications; counter-indications; animal study data; clinical study data; and/or references. The instructions may be printed directly on the container (when present), or as a label applied to the container, or as a separate sheet, pamphlet, card, or folder supplied in or with the container.

### Subject Monitoring

The disease state or treatment of a subject having breast cancer, or a propensity to develop metastatic breast cancer can be monitored using the methods and compositions of the invention. In one embodiment, the expression of markers, including Twist, CD44, CD24, ABCC1, ABCG2, ALDH, CD10, CD133, choline kinase, CXCR4, lactate dehydrogenase, Lin⁻, PROCR, ER, ESA, MUC1, and p27 present in a bodily fluid, such as nipple aspirant, ductal lavage, or milk is monitored. Such monitoring may be useful, for example, in assessing the efficacy of a particular drug in a subject or in assessing disease progression. Therapeutics that increase or decrease the expression of a marker of the invention (e.g., Twist, CD44, CD24, ABCC1, ABCG2, ALDH, CD10, CD133, choline kinase, CXCR4, lactate dehydrogenase, Lin⁻, PROCR, ER, ESA, MUC1, and p27) so as to reduce or eliminate the Twist⁺/CD44⁺/CD24^{-/low} cell sub-population are taken as particularly useful in the invention.

### Types of Biological Samples

The level of Twist, CD44, CD24, ABCC1, ABCG2, ALDH, CD10, CD133, choline kinase, CXCR4, lactate dehydrogenase, Lin⁻, PROCR, ER, ESA, MUC1, and p27 protein or polynucleotide is measured in different types of biologic samples. In one embodiment, the level of Twist, CD44, and CD24 proteins or polynucleotides is measured in different types of biologic samples. In another embodiment, the level of Twist, CD44, CD24, and any one or more of ABCC1, ABCG2, ALDH, CD10, CD133, choline kinase, CXCR4, lactate dehydrogenase, Lin⁻, PROCR, ER, ESA, MUC1, and p27 proteins or polynucleotides is measured in different types of biologic samples. In one embodiment, the biologic sample is a tissue sample that includes cells of a tissue or organ (e.g., breast cells). Breast cell tissue is obtained, for example, from a biopsy of the breast. In another embodiment, the biologic sample is a biologic fluid sample (e.g., nipple aspirant, ductal lavage, milk).

### Diagnostic Assays

The present invention provides a number of diagnostic assays that are useful for the identification or characterization of breast cancer, or a propensity to develop metastatic breast cancer. In one embodiment, breast cancer is characterized by quantifying the level of one or more of the following markers: Twist, CD44, CD24, ABCC1, ABCG2, ALDH, CD10, CD133, choline kinase, CXCR4, lactate dehydrogenase, Lin⁻, PROCR, ER, ESA, MUC1, and p27. In other embodiments, the cancer is further characterized as lacking estrogen receptor(s) (ER), a human epidermal growth factor receptor 2 (Her2), and/or a progesterone receptor(s) (PR). While the examples provided below describe specific methods of detecting levels of these markers, the skilled artisan appreciates that the invention is not limited to such methods. Marker levels are quantifiable by any standard method; such methods include, but are not limited to real-time PCR, Southern blot, PCR, mass spectroscopy, and/or antibody binding.

The examples describe primers used in the invention for amplification of markers of the invention. The primers of the invention embrace oligonucleotides of sufficient length and appropriate sequence so as to provide specific amplification. While exemplary primers are provided herein, it is understood that any primer that hybridizes with the marker sequences of the invention are useful in the methods of the invention for detecting marker levels.

The level of any two or more of the markers described herein defines the marker profile of a breast cancer. The level of marker is compared to a reference. In one embodiment, the reference is the level of marker present in a control sample obtained from a patient that does not have breast cancer or from a patient that does not have metastatic disease. In another embodiment, the reference is a baseline level of marker present in a biologic sample derived from a patient prior to, during, or after treatment for a breast cancer. In yet another embodiment, the reference is a standardized curve. The level of any one or more of the markers described herein (e.g., the combination of Twist, CD44, and CD24; the combination of Twist, CD44, and CD24, and any one or more of ABCC1, ABCG2, ALDH, CD10, CD133, choline kinase, CXCR4, lactate dehydrogenase, Lin⁻, PROCR, ER, ESA, MUC1, and p27) is used, alone or in combination with other standard methods, to determine the stage or grade of a breast cancer or tumor.

For example, a subject sample may be characterized not only for marker expression but also for tumor grade. Grading may be used to describe how abnormal or aggressive the neoplastic cells appear, while staging is used to describe the extent of the neoplasia. The grade and stage of the neoplasia is indicative of the patient's long-term prognosis (i.e., probable response to treatment and survival). Thus, the methods of the invention are useful for predicting a patient's prognosis, and for selecting a course of treatment.

There are several different systems for grading cancer. The Bloom-Richardson system is commonly used for grading breast cancer, and has a scale of I-III. A pathologist will take a sample of tissue from a tumor, and examine it under a microscope. Tumor cells that look most like normal cells are given a low grade, while those that look the most abnormal are given a high grade. High-grade tumors are fast-growing, metastatic, and aggressive. A pathologist looks at the tumor cells and checks for three microscopic features: 1) degree of tumor tubule formation; 2) tumor mitotic activity (i.e. rate of cell division); and 3) nuclear grade (cell size and uniformity). Each feature is scored on a scale of 1-3. The score of all three features are added together for a total between 3 and 9, where a score of 3-5 is Grade I, a score of 6-7 is Grade II, and a score of 8-9 is Grade III. This grading system relies heavily on the judgment of the pathologist, hence different pathologists are likely to score the same tissue sample differently. Thus, the methods of the invention are useful for predicting a grade of cancer more accurately and consistently.

### Selection of a Treatment Method

After a subject is diagnosed as having breast cancer a method of treatment is selected. In breast cancer, for example, a number of standard treatment regimens are available. The marker profile of the cancer is used in selecting a treatment method. In one embodiment, less aggressive cancers have undetectable levels of cells that are Twist⁺/CD44⁺/CD24^{-/low}. In contrast, breast cancers having increased numbers of Twist⁺/CD44⁺/CD24^{-/low} cells correlate with a poor clinical outcome, such as metastasis or death. Accordingly, the presence of Twist⁺/CD44⁺/CD24^{-/low} cells is indicative that aggressive therapy should be selected and the absence of Twist⁺/CD44⁺/CD24^{-/low} cells is indicative that less aggressive therapy should be selected. In one embodiment, the method further comprises detecting estrogen receptor (ER), a human epidermal growth factor receptor 2 (Her2), and/or a progesterone receptor (PR) in the sample. Patients identified as having aggressive cancers are likely to benefit from a combination of Twist inhibitory nucleic acid molecule (e.g., an siRNA, shRNA, or antisense RNA), a methylation inhibitor (e.g., 5-azacytidine, 5-azadeoxycytidine, procainamide, zebularine, and RG108), and an HDAC inhibitor (e.g., valproic acid, sodium butyrate, and Trichostatin A).

Less aggressive breast cancers are likely to be susceptible to less aggressive treatment methods. Where a breast cancer sample includes Twist⁺/CD44⁺/CD24^{-/low} cells, the breast cancer is identified as requiring aggressive intervention. Aggressive therapeutic regimens typically include one or more of the following therapies: radical mastectomy, radiation therapy (e.g., external beam and brachytherapy), hormone therapy, and high dose chemotherapies.

### Patient Monitoring

The diagnostic methods of the invention are also useful for monitoring the course of a breast cancer in a patient or for assessing the efficacy of a therapeutic regimen. In one embodiment, the diagnostic methods of the invention are used periodically to monitor the polynucleotide or polypeptide levels of Twist, CD44, and CD24, and any one or more of ABCC1, ABCG2, ALDH, CD10, CD133, choline kinase, CXCR4, lactate dehydrogenase, Lin⁻, PROCR, ER, ESA, MUC1, and p27. In one example, the breast cancer is characterized using a diagnostic assay of the invention prior to administering therapy. This assay provides a baseline that describes the level of one or more markers of the cancer prior to treatment. Additional diagnostic assays are administered during the course of therapy to monitor the efficacy of a selected therapeutic regimen. A therapy is identified as efficacious when a diagnostic assay of the invention detects a decrease in Twist⁺/CD44⁺/CD24^{-/low} cell sub-populations relative to the baseline level prior to treatment.

In one embodiment, the invention provides a method of monitoring treatment progress. The method includes the step of determining a level of diagnostic markers (Markers) (e.g., Twist, CD44, and CD24, and any one or more of ABCC1, ABCG2, ALDH, CD10, CD133, choline kinase, CXCR4, lactate dehydrogenase, Lin⁻, PROCR, ER, ESA, MUC1, and p27) or diagnostic measurement (e.g., screen, assay) in a subject suffering from or susceptible to breast cancer, in which the subject has been administered a therapeutic amount of a compound herein sufficient to treat the disease or symptoms thereof. The level of Markers determined in the method can be compared to known levels of Markers in either healthy normal controls or in other afflicted patients to establish the subject's disease status. In certain preferred embodiments, a pre-treatment level of Marker in the subject is determined prior to beginning treatment according to this invention; this pre-treatment level of Markers can then be compared to the level of Markers in the subject after the treatment commences, to determine the efficacy of the treatment.

### Therapy

Therapy may be provided wherever cancer therapy is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of cancer being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest cancer cells that may have spread to other parts of the body from the original tumor, to relieve symptoms caused by the cancer, or to prevent cancer in the first place.

As used herein, the term “breast cancer” is meant a cancer originating from breast tissue, most commonly from the inner lining of milk ducts or the lobules that supply the ducts with milk. Cancer growth is uncontrolled and progressive, and occurs under conditions that would not elicit, or would cause cessation of, multiplication of normal cells.

### Screening Methods

The invention provides methods for identifying agents useful for the treatment or prevention of breast cancer. Screens for the identification of such agents employ breast cancer stem cells identified according to the methods of the invention. The use of such cells, which express Twist⁺/CD44⁺/CD24^{-/low}, is particularly advantageous for the identification of agents that reduce the survival of this aggressive subpopulation of breast cancer cells. Agents identified as reducing the survival, reducing the proliferation, or increasing cell death of Twist⁺/CD44⁺/CD24^{-/low} subpopulations are particularly useful. By “reducing cell survival” is meant negatively altering cell viability. In one embodiment, methods that reduces cell survival creates a corresponding increase in cell death. Assays for measuring cell viability are known in the art, and are described, for example, by Crouch et al. (J. Immunol. Meth, 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Assays for measuring cell death are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

Cells of the invention, i.e., cell populations enriched for breast cancer initiating cells, are particularly useful in the aforementioned screening methods. Agents that reduce the proliferation or survival of such cells are useful for the treatment of breast cancer, including metastatic disease. Methods for isolating such cells using the markers delineated herein are known in the art and described herein (see the Examples). For example, stem cells can be separated or concentrated by a variety of procedures known to those of skill in the art including, but are not limited to, magnetic separation (e.g. using antibody-coated magnetic beads), affinity chromatography, cytotoxic agents (e.g. either joined to a monoclonal antibody or used with complement), and “panning,” which uses a monoclonal antibody attached to a solid matrix. Antibodies attached to solid matrices, such as magnetic beads, agarose beads, polystyrene beads, and follow fiber membranes and plastic surfaces, allow for direct separation of cells. Cells bound by an antibody can be removed, isolated, and/or concentrated by physically separating the solid support from the cell suspension. The exact conditions used for a cell isolation or separation procedure depend on factors specific to the given cell type and the procedure employed. The selection of appropriate conditions is well within the ability of one of skill in the art. Methods of cell separation and purification are found in U.S. Pat. No. 5,888,499, which is expressly incorporated by reference.

In one embodiment, antibodies can be conjugated to biotin or fluorochromes, which can be used to separate stem cells and or non-stem cells. For example, antibodies conjugated to a fluorochrome can be sorted in a fluorescence activated cell sorter (FACS) machine. Alternatively, antibodies conjugated to biotin may be bound to a solid support, which can then be isolated, and the cells can be subsequently removed with avidin or streptavidin.

Any technique may be employed as long as it is not detrimental to the viability of the desired cells.

In a preferred embodiment, breast cancer stem cells can be separated in an automated closed system such as the Nexell Isolex 300i Magnetic Cell Selection System. Generally, this is done to maintain sterility and to insure standardization of the cell separation methodology.

Once purified or concentrated the cells may be aliquoted and frozen, preferably, in liquid nitrogen or used immediately as described below. Frozen cells may be thawed and used as needed. Cryoprotective agents that can be used to store cells include, but are not limited to, dimethyl sulfoxide (DMSO) (Lovelock, J. E. and Bishop, M. W. H., 1959, Nature 183:1394-1395; Ashwood-Smith, M. J., 1961, Nature 190:1204-1205), hetastarch, glycerol, polyvinylpyrrolidine (Rinfret, A. P., 1960, Ann. N.Y. Acad. Sci. 85:576), polyethylene glycol (Sloviter, H. A. and Ravdin, R. G., 1962, Nature 196:548), albumin, dextran, sucrose, ethylene glycol, i-erythritol, D-ribitol, D-mannitol (Rowe, A. W., et al., 1962, Fed. Proc. 21:157), D-sorbitol, i-inositol, D-lactose, choline chloride (Bender, M. A., et al., 1960, J. Appl. Physiol. 15:520), amino acids (Phan The Tran and Bender, M. A, 1960, Exp. Cell Res. 20:851), methanol, acetamide, glycerol monoacetate (Lovelock. J. E., 1954, Biochem. J. 56:265), and inorganic salts (Phan The Tran and Bender, M. A., 1960, Proc. Soc. Exp. Biol. Med. 104:388; Phan The Tran and Bender, M. A., 1961, in Radiobiology Proceedings of the Third Australian Conference on Radiobiology, Ilbery, P. L. T., ed., Butterworth, London, p. 59). Typically, the cells may be stored in 10% DMSO, 50% serum, and 40% RPMI 1640 medium.

In a preferred embodiment, the isolated breast cancer stem cells can be washed to remove serum proteins and soluble blood components, such as auto-antibodies, inhibitors, etc., using techniques well known in the art. Generally, this involves the addition of physiological media or buffer, followed by centrifugation. This may be repeated as necessary. Isolated stem cells can be resuspended in physiological media, for example phenol-red free minimal essential media (PRF-MEM) containing 5% charcoal stripped serum (CSS). Other physiological media known to those of skill in the art can also be used. Generally, the cells are then counted.

In specific embodiments, the purified or enriched population of cells is about 50 to about 55%, about 55 to about 60%, about 65 to about 70%, about 70 to about 75%, about 75 to about 80%, about 80 to about 85%, about 85 to about 90%, about 90 to about 95% or about 95 to about 100% of the cells in the composition.

Methods of observing changes in the level, interactions, or activity of Twist, CD44, and CD24, and any one or more of ABCC1, ABCG2, ALDH, CD10, CD133, choline kinase, CXCR4, lactate dehydrogenase, Lin⁻, PROCR, ER, ESA, MUC1, and p27 are exploited in high throughput assays for the purpose of identifying compounds that modulate Twist⁺/CD44⁺/CD24^{-/low} subpopulations, e.g., transcriptional regulation or protein-nucleic acid interactions. For example, compounds that inhibit Twist to a regulated gene (e.g., but not limited to, ER, CD24, and p27), or that inhibit another biological activity, may be identified by such assays. In addition, compounds that modulate the expression of a Twist, CD44, CD24, ABCC1, ABCG2, ALDH, CD10, CD133, choline kinase, CXCR4, lactate dehydrogenase, Lin⁻, PROCR, ER, ESA, MUC1, and p27 polypeptide or nucleic acid molecule whose expression is altered in a patient having a cancer may be identified.

Any number of methods are available for carrying out screening assays to identify new candidate compounds that decrease the expression of one or more of these markers. In one example, candidate compounds are added at varying concentrations to the culture medium of cultured cells expressing one of the nucleic acid sequences of the invention. Gene expression is then measured, for example, by microarray analysis, Northern blot analysis (Ausubel et al., supra), or RT-PCR, using any appropriate fragment prepared from the nucleic acid molecule as a hybridization probe. The level of gene expression in the presence of the candidate compound is compared to the level measured in a control culture medium lacking the candidate molecule. A compound which reduces the expression of, for example, a Twist or CD44 gene, or a functional equivalent thereof, is considered useful in the invention; such a molecule may be used, for example, as a therapeutic to treat a breast cancer in a human patient.

In another example, the effect of candidate compounds may be measured at the level of polypeptide production using the same general approach and standard immunological techniques, such as Western blotting or immunoprecipitation with an antibody specific for a polypeptide encoded by an Twist, CD44, CD24, ABCC1, ABCG2, ALDH, CD10, CD133, choline kinase, CXCR4, lactate dehydrogenase, Lin⁻, PROCR, ER, ESA, MUC1, and p27 gene. For example, immunoassays may be used to detect or monitor the expression of at least one of the polypeptides of the invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In some embodiments, a compound that promotes an increase in the expression or biological activity of the polypeptide is considered particularly useful. Again, such a molecule may be used, for example, as a therapeutic to delay, ameliorate, or treat a cancer or neoplasia in a human patient.

In yet another working example, candidate compounds may be screened for those that specifically bind to a polypeptide encoded by a Twist, CD44, CD24, ABCC1, ABCG2, ALDH, CD10, CD133, choline kinase, CXCR4, lactate dehydrogenase, Lin⁻, PROCR, ER, ESA, MUC1, or p27 gene. The efficacy of such a candidate compound is dependent upon its ability to interact with such a polypeptide or a functional equivalent thereof. Such an interaction can be readily assayed using any number of standard binding techniques and functional assays (e.g., those described in Ausubel et al., supra). In one embodiment, a candidate compound may be tested in vitro for its ability to specifically bind a polypeptide of the invention. In another embodiment, a candidate compound is tested for its ability to inhibit the biological activity of a polypeptide described herein, such as a Twist, CD44, CD24, ABCC1, ABCG2, ALDH, CD10, CD133, choline kinase, CXCR4, lactate dehydrogenase, Lin⁻, PROCR, ER, ESA, MUC1, or p27 polypeptide. The biological activity of an Twist, CD44, CD24, ABCC1, ABCG2, ALDH, CD10, CD133, choline kinase, CXCR4, lactate dehydrogenase, Lin⁻, PROCR, ER, ESA, MUC1, or p27 polypeptide may be assayed using any standard method, for example, a matrigel cell invasion or cell migration assay.

In another working example, a nucleic acid described herein (e.g., a Twist, CD44, CD24, ABCC1, ABCG2, ALDH, CD10, CD133, choline kinase, CXCR4, lactate dehydrogenase, Lin⁻, PROCR, ER, ESA, MUC1, and p27 nucleic acid) is expressed as a transcriptional or translational fusion with a detectable reporter, and expressed in an isolated cell (e.g., mammalian) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that alters the expression of the detectable reporter is a compound that is useful for the treatment of a breast cancer. Preferably, the compound decreases the expression of the reporter.

In another example, a candidate compound that binds to a polypeptide encoded by a Twist, CD44, CD24, ABCC1, ABCG2, ALDH, CD10, CD133, choline kinase, CXCR4, lactate dehydrogenase, Lin⁻, PROCR, ER, ESA, MUC1, or p27 gene may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the Twist, CD44, CD24, ABCC1, ABCG2, ALDH, CD10, CD133, choline kinase, CXCR4, lactate dehydrogenase, Lin⁻, PROCR, ER, ESA, MUC1, or p27 polypeptide is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to increase the activity of an Twist, CD44, CD24, ABCC1, ABCG2, ALDH, CD10, CD133, choline kinase, CXCR4, lactate dehydrogenase, Lin⁻, PROCR, ER, ESA, MUC1, or p27 polypeptide (e.g., as described herein). Compounds isolated by this approach may also be used, for example, as therapeutics to treat a cancer or neoplasia in a human patient. Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized.

Potential antagonists include organic molecules, peptides, peptide mimetics, polypeptides, nucleic acids, and antibodies that bind to a nucleic acid sequence or polypeptide of the invention (e.g., a Twist, CD44, CD24, ABCC1, ABCG2, ALDH, CD10, CD133, choline kinase, CXCR4, lactate dehydrogenase, Lin⁻, PROCR, ER, ESA, MUC1, or p27 polypeptide or nucleic acid molecule).

Each of the DNA sequences listed herein may also be used in the discovery and development of a therapeutic compound for the treatment of cancer. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

Optionally, compounds identified in any of the above-described assays may be confirmed as useful in an assay for compounds that modulate the propensity of a cancer to metastasize.

Small molecules of the invention preferably have a molecular weight below 2,000 daltons, more preferably between 300 and 1,000 daltons, and most preferably between 400 and 700 daltons. It is preferred that these small molecules are organic molecules.

Methods of the invention are useful for the high-throughput low-cost screening of candidate agents useful for the treatment or prevention of breast cancer. In one embodiment, a candidate agent is an agent that specifically binds to Twist and inhibits it's repression of downstream target genes. One skilled in the art appreciates that the effects of a candidate agent on a cell is typically compared to a corresponding control cell not contacted with the candidate agent. Thus, the screening methods include comparing a biomarker expression profile of a cell contacted by a candidate agent to that of an untreated control cell.

In other embodiments, the expression or activity of Twist in a cell treated with a candidate agent is compared to untreated control samples to identify a candidate compound that increases the expression or activity of Twist target genes in the contacted cell. Polypeptide expression or activity can be compared by procedures well known in the art, such as Western blotting, flow cytometry, immunocytochemistry, binding to magnetic and/or Twist-specific antibody-coated beads, in situ hybridization, fluorescence in situ hybridization (FISH), ELISA, microarray analysis, RT-PCR, Northern blotting, or colorimetric assays, such as the Bradford Assay and Lowry Assay.

In one working example, one or more candidate agents are added at varying concentrations to the culture medium containing a Twi⁺/CD44⁺/CD24^{-/low} sub-population. An agent that promotes the repression of Twist and/or it's activity is considered useful in the invention; such an agent may be used, for example, as a therapeutic to prevent, delay, ameliorate, or stabilize the formation of BCICs within a cell population. Once identified, agents of the invention (e.g., agents that specifically bind to and/or inhibit Twist) may be used to decrease or prevent the formation of BCICs. An agent identified according to a method of the invention can be locally or systemically delivered to, for example, a breast cancer tumor to decrease or prevent the formation of BCICs.

In one embodiment, the effect of a candidate agent may, in the alternative, be measured at the level of Twist polypeptide production using the same general approach and standard immunological techniques, such as Western blotting or immunoprecipitation with an antibody specific for Twist. For example, immunoassays may be used to detect or monitor the expression of Twist in a BCIC or other breast cell sub-population. In one embodiment, the invention identifies a polyclonal or monoclonal antibody (produced as described herein) that is capable of binding to and inhibiting a Twist polypeptide. A compound that promotes a decrease in the expression or activity of a Twist polypeptide is considered particularly useful. Again, such a molecule may be used, for example, as a therapeutic to combat breast cancer, or to prevent or treat a neoplasia.

Alternatively, or in addition, candidate compounds may be identified by first assaying for compounds that specifically bind to and inactivate a Twist polypeptide of the invention, and then testing their effect on the expression of downstream target genes regulated by Twist. In one embodiment, the efficacy of a candidate agent is dependent upon its ability to interact with the Twist polypeptide. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate target gene expression.

In one particular example, a candidate compound that binds to a Twist polypeptide may be identified using a chromatography-based technique. For example, a recombinant Twist polypeptide of the invention may be purified by standard techniques from cells engineered to express the polypeptide, or may be chemically synthesized, once purified the peptide is immobilized on a column. A solution of candidate agents is then passed through the column, and an agent that specifically binds the Twist polypeptide or a fragment thereof is identified on the basis of its ability to bind to Twist polypeptide and to be immobilized on the column. To isolate the agent, the column is washed to remove non-specifically bound molecules, and the agent of interest is then released from the column and collected. Agents isolated by this method (or any other appropriate method) may, if desired, be further purified (e.g., by high performance liquid chromatography). In addition, these candidate agents may be tested for their ability to modulate the expression of Twist target genes. Compounds that are identified as binding to a Twist polypeptide with an affinity constant less than or equal to 1 nM, 5 nM, 10 nM, 100 nM, 1 mM or 10 mM are considered particularly useful in the invention.

Such agents may be used, for example, as a therapeutic to combat the formation of BCICs. Optionally, agents identified in any of the above-described assays may be confirmed as useful in inhibiting Twist mediated gene repression.

Additionally, each of the polynucleotide sequences provided herein may also be used in the discovery and development of anti-breast cancer compounds.

### Test Extracts and Agents

In general, agents that modulate Twist, CD44, CD24, ABCC1, ABCG2, ALDH, CD10, CD133, choline kinase, CXCR4, lactate dehydrogenase, Lin⁻, PROCR, ER, ESA, MUC1, or p27 expression, biological activity, or Twist, CD44, CD24, ABCC1, ABCG2, ALDH, CD10, CD133, choline kinase, CXCR4, lactate dehydrogenase, Lin⁻, PROCR, ER, ESA, MUC1, or p27-dependent signaling are identified from large libraries of both natural products, synthetic (or semi-synthetic) extracts or chemical libraries, according to methods known in the art.

Those skilled in the art will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modifications of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from, for example, Brandon Associates (Merrimack, N.H.), Aldrich Chemical (Milwaukee, Wis.), and Talon Cheminformatics (Acton, Ont.)

Alternatively, libraries of natural compounds in the form of bacterial, fungal, plant, and animal extracts are commercially available from a number of sources, including, but not limited to, Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art (e.g., by combinatorial chemistry methods or standard extraction and fractionation methods). Furthermore, if desired, any library or compound may be readily modified using standard chemical, physical, or biochemical methods.

### Combination Therapies

The present invention provides therapeutic compositions and methods for the treatment of breast cancer, which may be used alone or in combination with any other cancer therapy known in the art. In particular, the invention provides agents (e.g., small compounds, polypeptides, polynucleotides) that inhibit the biological activity of any one or more of Twist, CD44, CD24, ABCC1, ABCG2, ALDH, CD10, CD133, choline kinase, CXCR4, lactate dehydrogenase, Lin, PROCR, ER, ESA, MUC1, or p27 expression or biological activity. In one particular embodiment, the invention provides inhibitory nucleic acids that inhibit the expression of any one or more of Twist, CD44, CD24, ABCC1, ABCG2, ALDH, CD10, CD133, choline kinase, CXCR4, lactate dehydrogenase, Lin, PROCR, ER, ESA, MUC1, or p27. Agents of the invention may be administered alone or in any combination that is effective to treat breast cancer. If desired, agents of the invention are administered in combination with any other standard cancer therapy; such methods are known to the skilled artisan (e.g., Wadler et al., Cancer Res. 50:3473-86, 1990), and include, but are not limited to, chemotherapy, hormone therapy, immunotherapy (include, but are not limited to, immunotherapy that will specifically target cancer stem cell transcription factors), radiotherapy, and any other therapeutic method used for the treatment of cancer.

In general, Twist agonists (e.g., agents that specifically bind and inhibit a Twist polypeptide) are identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include known those known as therapeutics for the treatment of pathogen infections. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have Twist binding and/or inhibiting activity, further fractionation of the positive lead extract is necessary to isolate molecular constituents responsible for the observed effect. Thus, the goal of the extraction, fractionation, and purification process is the careful characterization and identification of a chemical entity within the crude extract that Twist or that inhibits BCIC formation and/or proliferation. Methods of fractionation and purification of such heterogenous extracts are known in the art. If desired, compounds shown to be useful as therapeutics are chemically modified according to methods known in the art.

### Pharmaceutical Therapeutics

The invention provides a simple means for identifying compositions (including nucleic acids, peptides, small molecule inhibitors, and mimetics) capable of binding to and inhibiting Twist. Accordingly, a chemical entity discovered to have medicinal value using the methods described herein is useful as a drug or as information for structural modification of existing compounds, e.g., by rational drug design. Such methods are useful for screening agents having an effect on a variety of conditions characterized by a reduction in innate immunity. For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the pathogen infection or neoplasia. Generally, amounts will be in the range of those used for other agents used in the treatment of other types of cancer or neoplasia, although in certain instances lower amounts will be needed because of the increased specificity of the compound. A compound is administered at a dosage that inhibits Twist, or that decreases or prevents BCIC formation or proliferation as determined by a method known to one skilled in the art, or using any that assay that measures the expression or the biological activity of a Twist polypeptide.

### Inhibitory Nucleic Acids

Inhibitory nucleic acid molecules are those oligonucleotides that inhibit the expression or activity of a polypeptide of interest (e.g. Twist, ER, CD44, or CD24). Such oligonucleotides include single and double stranded nucleic acid molecules (e.g., DNA, RNA, and analogs thereof) that bind a nucleic acid molecule that encodes a Twist polypeptide (e.g., antisense molecules, siRNA, shRNA) as well as nucleic acid molecules that bind directly to a Twist polypeptide to modulate its biological activity (e.g., aptamers).

Ribozymes

Catalytic RNA molecules or ribozymes that include an antisense Twist sequence of the present invention can be used to inhibit expression of a Twist nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002). More specifically, the therapeutic effectiveness of an siRNA approach in humans was demonstrated in vivo by Davis et al. (Nature 464(7291):1067-70. 2010).

Given the sequence of a target gene, siRNAs may be designed to inactivate that gene. Such siRNAs, for example, could be administered directly to an affected tissue, or administered systemically. The nucleic acid sequence of a Twist gene can be used to design small interfering RNAs (siRNAs). The 21 to 25 nucleotide siRNAs may be used, for example, as therapeutics to treat a vascular disease or disorder.

The inhibitory nucleic acid molecules of the present invention may be employed as double-stranded RNAs for RNA interference (RNAi)-mediated knock-down of Twist expression. In one embodiment, Twist expression is reduced in an endothelial cell or an astrocyte. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

Oligonucleotides and other Nucleobase Oligomers

At least two types of oligonucleotides induce the cleavage of RNA by RNase H: polydeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed, including covalently-closed multiple antisense (CMAS) oligonucleotides (Moon et al., Biochem J. 346:295-303, 2000; PCT Publication No. WO 00/61595), ribbon-type antisense (RiAS) oligonucleotides (Moon et al., J. Biol. Chem. 275:4647-4653, 2000; PCT Publication No. WO 00/61595), and large circular antisense oligonucleotides (U.S. Patent Application Publication No. US 2002/0168631 A1).

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

Specific examples of preferred nucleobase oligomers useful in this invention include oligonucleotides containing modified backbones or non-natural internucleoside linkages. As defined in this specification, nucleobase oligomers having modified backbones include those that retain a phosphorus atom in the backbone and those that do not have a phosphorus atom in the backbone. For the purposes of this specification, modified oligonucleotides that do not have a phosphorus atom in their inter-nucleoside backbone are also considered to be nucleobase oligomers.

Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriest-ers, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl inter-nucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other nucleobase oligomers, both the sugar and the internucleoside linkage, i.e., the backbone, are replaced with novel groups. The nucleobase units are maintained for hybridization with an IAP. One such nucleobase oligomer, is referred to as a Peptide Nucleic Acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular —CH₂₋NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and —O—N(CH₃)—CH₂—CH₂—. In other embodiments, the oligonucleotides have morpholino backbone structures described in U.S. Pat. No. 5,034,506.

Nucleobase oligomers may also contain one or more substituted sugar moieties. Nucleobase oligomers comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly preferred are O[(CH₂)ₙO]ₙCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred nucleobase oligomers include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Preferred modifications are 2′-O-methyl and 2′-methoxyethoxy (2′-O—CH₂CH₂OCH₃, also known as 2′-O-(2-methoxyethyl) or 2′-MOE). Another desirable modification is 2′-dimethylaminooxyethoxy (i.e., O(CH₂)₂ON(CH₃)₂), also known as 2′-DMAOE. Other modifications include, 2′-aminopropoxy (2′-OCH₂CH₂CH₂NH₂) and 2′-fluoro (2′-F). Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleobase oligomers may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of a nucleobase oligomer of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-1060, 1994), a thioether, e.g., hexyl-5-tritylthiol (Manoharan et al., Ann. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-1118, 1991; Kabanov et al., FEBS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim. Biophys. Acta, 1264:229-237, 1995), or an octadecylamine or hexylamino-carbonyl-oxycholesterol moiety (Crooke et al., J. Pharmacol. Exp. Ther., 277:923-937, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes nucleobase oligomers that are chimeric compounds. “Chimeric” nucleobase oligomers are nucleobase oligomers, particularly oligonucleotides, that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleobase oligomers used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The nucleobase oligomers of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

A nucleobase oligomer of the invention, or other negative regulator of the Twist polypeptide, may be administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for IAP modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. The preferred dosage of a nucleobase oligomer of the invention is likely to depend on such variables as the type and extent of the disorder, the overall health status of the particular patient, the formulation of the compound excipients, and its route of administration.

As described above, if desired, treatment with a nucleobase oligomer of the invention may be combined with therapies for the treatment of proliferative disease (e.g., radiotherapy, surgery, or chemotherapy).

For any of the methods of application described above, a nucleobase oligomer of the invention is desirably administered intravenously or is applied to the site of the needed apoptosis event (e.g., by injection).

### Formulation of Pharmaceutical Compositions

The administration of a compound for the treatment of breast cancer or neoplasia may be by any suitable means that results in a concentration of the therapeutic that, combined with other components, is effective in ameliorating, reducing, or stabilizing a breast cancer or neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the breast; (v) formulations that allow for convenient dosing, such that doses are administered, for example, once every one or two weeks; and (vi) formulations that target a breast cancer or neoplasia by using carriers or chemical derivatives to deliver the therapeutic agent to a particular cell type (e.g., breast tissue). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a pathogen infection or neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active breast cancer therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in the form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms For Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active a anti-pathogen or anti-neoplasia therapeutic substance). The coating may be applied on the solid dosage form in a similar manner as that described in Encyclopedia of Pharmaceutical Technology, supra.

At least two anti-breast cancer or anti-neoplasia therapeutics may be mixed together in the tablet, or may be partitioned. In one example, the first active anti-breast cancer or anti-neoplasia therapeutic is contained on the inside of the tablet, and the second active anti-breast cancer or anti-neoplasia therapeutic is on the outside, such that a substantial portion of the second active anti-breast cancer or anti-neoplasia therapeutic is released prior to the release of the first active anti-breast cancer or anti-neoplasia therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active anti-breast cancer or anti-neoplasia therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Combination Therapies

Optionally, anti-breast cancer or anti-neoplasia therapeutic may be administered in combination with any other standard anti-breast cancer or anti-neoplasia therapy; such methods are known to the skilled artisan and described in Remington's Pharmaceutical Sciences by E. W. Martin. Kits

The invention provides kits for the identification or diagnosis of breast cancer, particularly breast cancer with Twist⁺/CD44⁺/CD24^{-/low} cell subpopulations. In some embodiments, the kit comprises a sterile container which contains a nucleic acid or antibody composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments. If desired a nucleic acid or antibody composition is provided together with instructions for identifying or diagnosing a subject having or at risk of developing breast cancer. The instructions will generally include information about the use of the compositions for the diagnosis of breast cancer. In other embodiments, the instructions include at least one of the following: description of the diagnostic agent; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The present invention provides methods of treating disease and/or disorders or symptoms thereof which comprise administering a therapeutically effective amount of a pharmaceutical composition to a subject (e.g., a mammal such as a human). Thus, one embodiment is a method of treating a subject suffering from or susceptible to breast cancer. The method includes the step of administering to the mammal a therapeutic amount of an amount of a compound herein sufficient to treat the cancer.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “risk” can be made by any objective or subjective determination by a diagnostic test or opinion of a subject or health care provider (e.g., genetic test, enzyme or protein marker, Marker (as defined herein), family history, and the like).

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to breast cancer, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

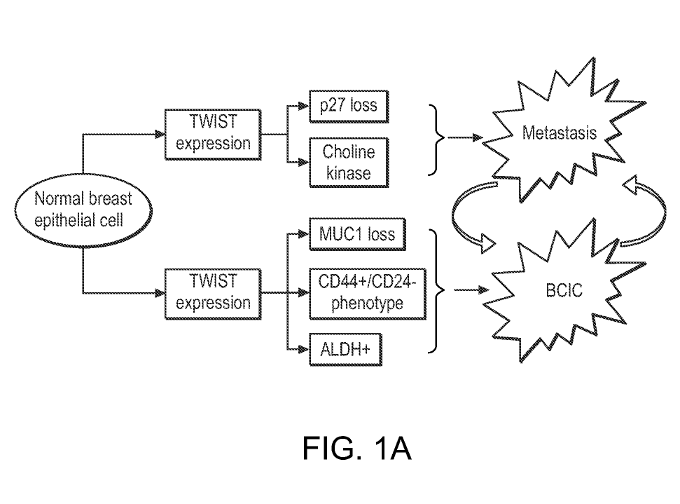

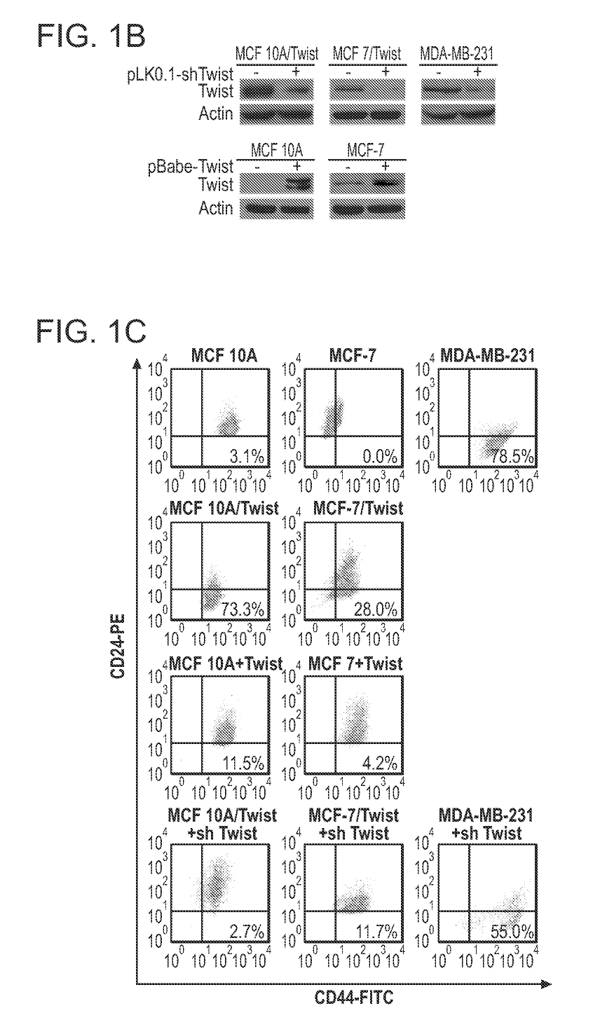

The Examples described below broadly relate to a cancer model whereby the overexpression of Twist results in the induction of epithelial mesenchymal transition (EMT), increased motility/invasion, and neovascularization and facilitates the generation of breast cancer initiating cells (BCICs) (FIG. 1A).

### Example 1

### A CD44⁺/CD24^{-/low} Subpopulation is Evident in Twist-Overexpressing Breast Cells

Recent studies have demonstrated that the cell surface expression pattern of CD44 and CD24 constitutes a major identifying marker of breast cancer stem cells. To determine the role of Twist in inducing the breast cancer stem cell subpopulation, Twist was overexpressed and knocked down in a panel of normal immortalized mammary cells and breast cancer cell lines. MCF-10A/Twist, MCF-7/Twist, and MDA-MB-231 cell lines were used as models for stable Twist overexpression as well as for transiently knocking down Twist expression using short hairpin RNA (shRNA) lentiviral constructs. Parental MCF-10A and MCF-7 cell lines were used as Twist-nonexpressing models into which Twist was transiently expressed using retroviral constructs. Immunoblot analysis was used to determine the levels of Twist expression in the transgenic cell lines as well as in Twist knockdown cell lines. A significant decrease in Twist expression was obtained in all three cell lines using shRNA lentiviral constructs (FIG. 1B), whereas empty vector controls did not alter the Twist expression levels in these cell lines.

The CD44⁺/CD24^{-/low} subpopulation was evaluated by flow cytometry in various parental cell lines (MCF-10A, MCF-7, MDAMB-231), transgenic cell lines (MCF-10A/Twist, MCF-7/Twist), Twist-overexpressing cell lines (MCF-10A-Twist and MCF-7-Twist), and in Twist knockdown breast cancer cell lines (MCF-10A/Twist-shTwist, MCF-7/Twist-shTwist, MDA-MB-231-shTwist).

As seen in FIG. 1C, the nontumorigenic cell lines MCF-10A (3.1%) and MCF-7 (0.0%) were observed to have a low degree of the CD44⁺/CD24^{-/low} subpopulation. This is in comparison to the higher percentage of CD44⁺/CD24^{-/low} subpopulation in the metastatic MDA-MB-231 cell line (78.5%) (top row). To evaluate if Twist-overexpressing breast cancer cells have altered CD44 and CD24 expression, multiple MCF-10A/Twist and MCF-7/Twist clones were analyzed for CD44 and CD24 expression. As seen in the second row, MCF-10A/Twist cells demonstrated increased CD44⁺/CD24^{-/low} (73.3%) compared with parental MCF-10A cells (3.1%). In addition, the MCF-7/Twist cell line exhibited a higher CD44⁺/CD24^{-/low} subpopulation (28.0%) compared with parental MCF-7 cells (0.0%). To confirm that the observed changes were caused by the effector functions of Twist and not caused by clonal selection, the CD44 and CD24 levels were determined in transient Twist-overexpressing breast cancer cell lines MCF-10A and MCF-7. As seen in the third row, MCF-10A and MCF-7 transduced with Twist showed an increase in the CD44⁺/CD24^{-/low} subpopulation (from 3.1% to 11.5% and from 0.0% to 4.2%, respectively).

### Example 2

### Knockdown of Twist Reverses the Stem Cell Phenotype

Because Twist overexpression altered CD44 and CD24 expression levels, it was determined whether decreasing Twist expression could reverse the cancer stem cell phenotype back to parental status. Toward this goal, Twist expression was knocked down in MCF-10A/Twist and MCF-7/Twist cell lines, using lentiviral-mediated shRNA knockdowns (FIG. 1B). These cell lines showed lowered Twist expression when compared with parental MCF-10A/Twist and MCF-7/Twist cells. Subsequent analysis of the CD44⁺/CD24^{-/low} subpopulation by flow cytometry revealed a reduction in the CD44⁺/CD24⁻ phenotype in both Twist knockdown cell lines-MCF-10A/Twist (reduced from 73.3% to 2.7%) and MCF-7/Twist (reduced from 28.0% to 11.7%; FIG. 1C, fourth row). Knockdown of Twist expression in MDA-MB-231 cells decreased the CD44⁺/CD24^{-/low} subpopulation from 78.5% in the parental cells to 55% in the knockdown cells (fourth row). Collectively, these data demonstrate that Twist is a major factor in modulating the CD44⁺/CD24^{-/low} subpopulation in breast cancer cells.

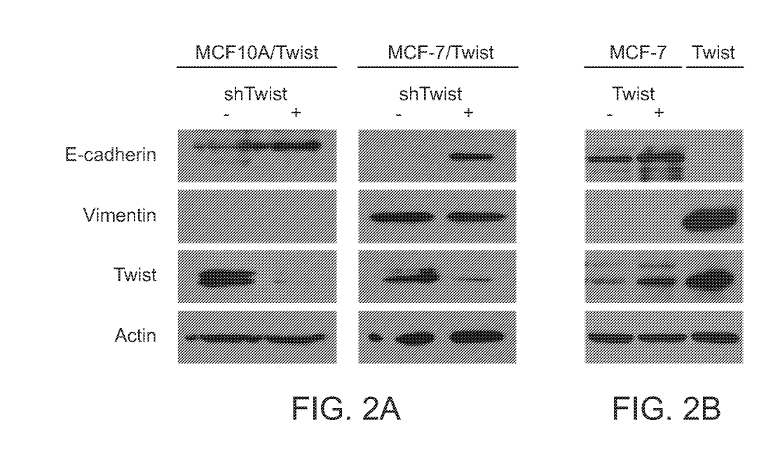

With respect to epithelial-mesenchymal transition (EMT) markers in Twist knockdown MCF-10A/Twist and MCF-7/Twist cells, an increase in E-cadherin levels, but little or no change in vimentin levels, was observed (FIG. 2A). Without wishing to be bound by theory, it is likely that Twist expression potentiates EMT and that down-regulating Twist reverses this phenotype, at least to some degree. Additionally, transient expression of Twist in MCF-7 cells was shown to alter CD44 and CD24 levels, but did not induce any change in protein expression of the EMT markers E-cadherin and vimentin (FIG. 2B). This is in contrast to high vimentin and low E-cadherin expression observed in stable Twist-expressing MCF-7 cells (FIG. 2B).

### Example 3

### MCF-7/Twist Cells Show Increased Efflux of Hoechst 33342 and Rhodamine 123 Dyes

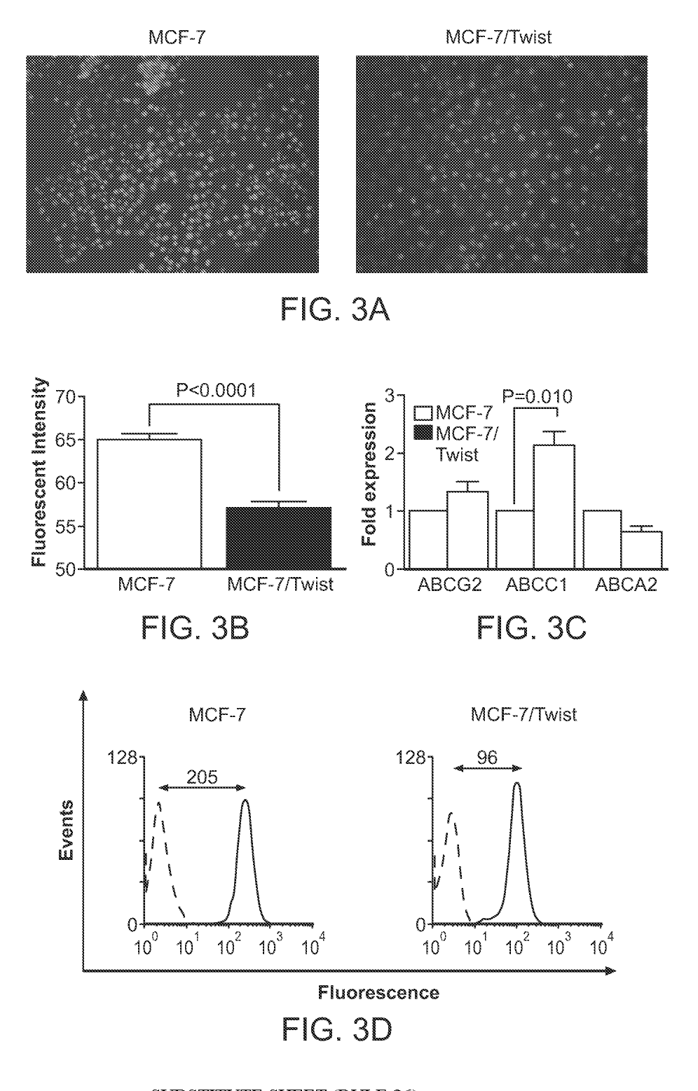

A characteristic of stem cells is increased drug resistance brought about by elevated expression of ATP-binding cassette (ABC) transporters on the cell surface. The functionality of these transporters can be characterized by studying the efflux of vital dyes such as Hoechst 33342 and Rhodamine 123 from treated cells. Because Twist expression also induces chemoresistance, the ability of MCF-7/Twist cells to extrude Hoechst 33342 dye microscopically was evaluated. As shown in FIGS. 3A and 3B, MCF-7/Twist cells retained significantly less dye compared with parental MCF-7 cells (relative fluorescence intensity per cell of 57 vs 65). To determine factors responsible for increased efflux, quantitative reverse transcription-PCR (qRT-PCR) was carried out for the expression of various stem cell factors such as ABC transporters ABCG2 and ABCC1 (MRP1). The results obtained revealed a significant increase in ABCC1 (MRP1) and a lesser increase in ABCG2 transcript levels in Twist overexpressing breast cancer cells (FIG. 3C).

Subsequently, the ability of MCF-7/Twist cells to extrude Rhodamine 123 dye was confirmed by analyzing mean fluorescence intensity as determined by flow cytometry. As seen in FIG. 3D, Rhodamine 123 was excluded more from the MCF-7/Twist cells than from the MCF-7 cells (96 vs 205). These results confirm that the chemoresistance of MCF-7/Twist cells is partially conferred by the overexpression of at least one of the transporter genes, ABCC1.

### Example 4

### MCF-7/Twist Cells Have Increased Aldehyde Dehydrogenase (ALDH) Activity

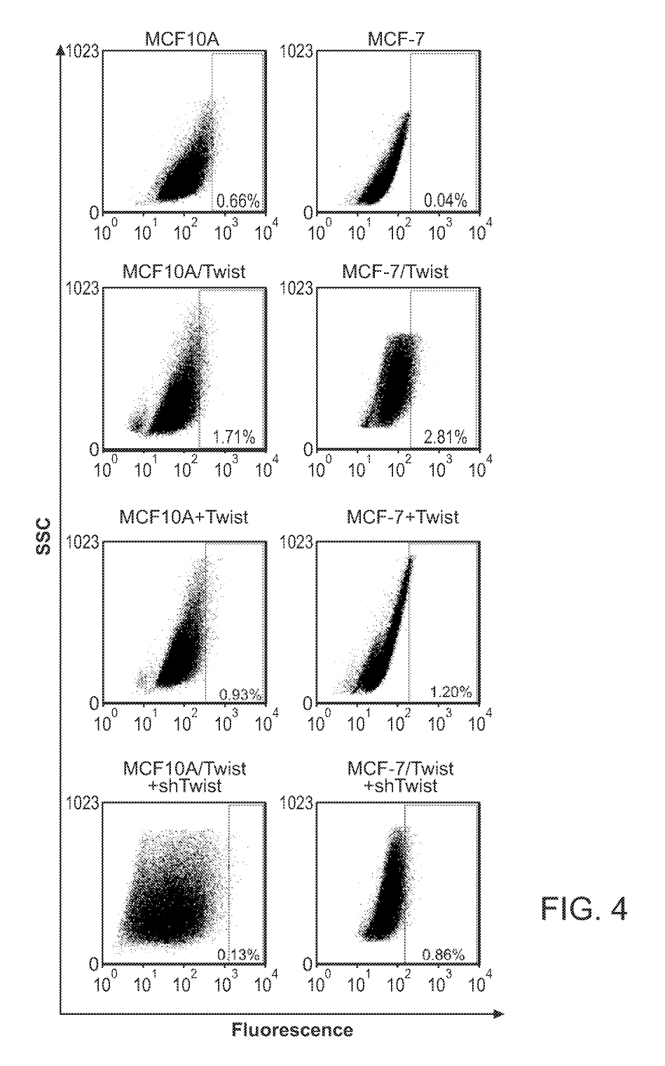

Expression of ALDH within the presumptive stem cell phenotype identifies and enriches self-renewing populations within the breast milieu. Given the initial finding that the expression of Twist in breast cells promotes the stem cell phenotype, ALDH expression was analyzed using the ALDEFLUOR assay (Stem Cell Technologies) in a panel of breast cancer cell lines as well as in MCF-10A/Twist and MCF-7/Twist transgenic cells. As shown in FIG. 4 (first row), the percentage of ALDH-positive cells was generally low in the breast cancer cell lines analyzed. However, stable expression of Twist increased the ALDH-positive cells from 0.66% to 1.71% in MCF-10A/Twist cells and from 0.04% to 2.81% in MCF-7/Twist cells (FIG. 4, second row). An increase in the ALDH-positive cells was also observed in MCF-10A cells (from 0.66% up to 0.93%) and MCF-7 cells (from 0.04% up to 1.2%) transduced with Twist-overexpressing retroviral constructs (FIG. 4, third row).

In further support of the role of Twist in altering ALDH activity, ALDH was estimated in Twist knockdown cell lines. As shown in FIG. 4 (fourth row), loss of Twist decreased the number of ALDH-positive cells in MCF-10A/Twist (from 1.71% down to 0.13%) and MCF-7/Twist (1.2% down to 0.86%). Interestingly, MDA-MB-231 has a very low subpopulation of ALDH⁺ cells (0.01%), which is not affected by Twist knockdown. Overall, these results support that Twist is a regulator of the breast cancer stem cell phenotype.

### Example 5

### Ability of Self-renewal of the CD44⁺/CD24^{-/low} Subpopulation in MCF 7/Twist Cells

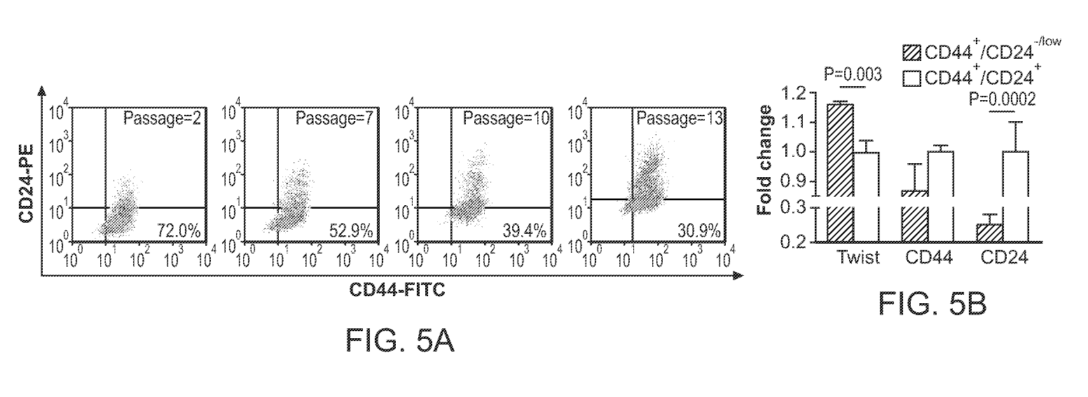

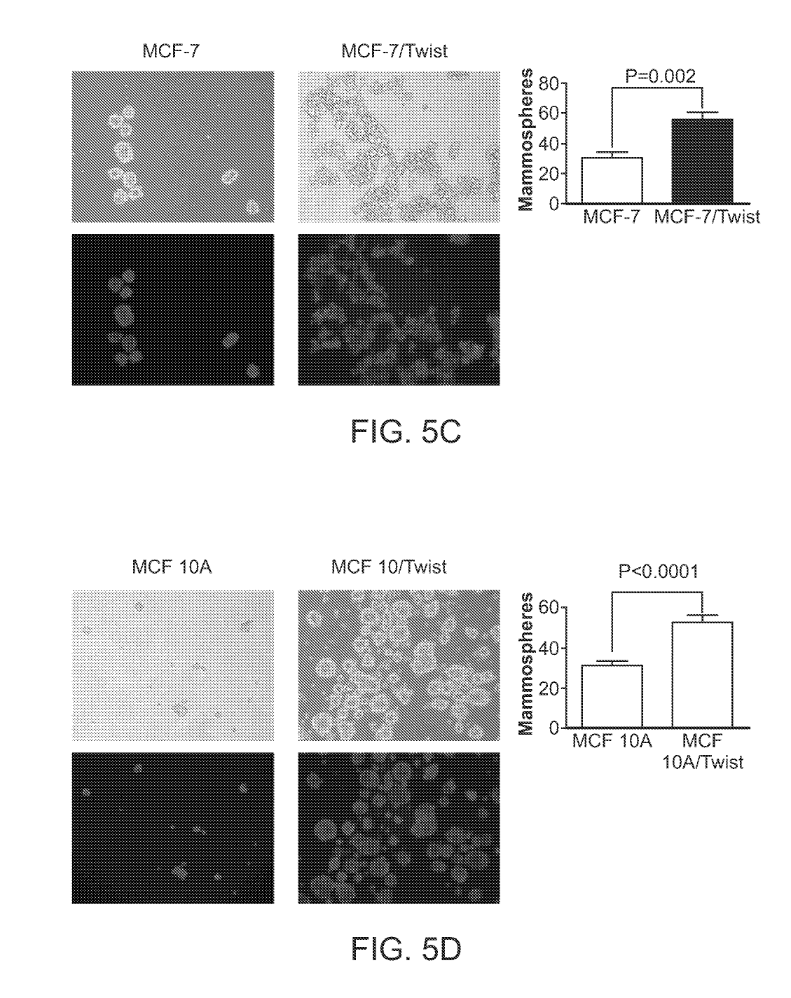

One of the defining characteristics of stem cells is the property of self-renewal, or asymmetric cell division. To ascertain whether the ability of asymmetric division is an inherent characteristic of Twist⁺ cells, the CD44⁺/CD24^{-/low} subpopulation was flow sorted from MCF-7/Twist cells using antibodies against CD44 and CD24. After purification, the enriched cells were allowed to divide in vitro, and the CD44⁺/CD24^{-/low} subpopulation was estimated at regular time intervals. The initial percentage of the purified subpopulation of CD44⁺/CD24^{-/low} cells was greater than 98%. As shown in FIG. 5A, continuous culture of the purified cells decreased the CD44⁺/CD24^{-/low} subpopulation from 72% (generation 2) to 31% (generation 13), which is an indication of self-renewal that characterizes the stem cell phenotype. After passage 15, the CD44⁺/CD24^{-/low} subpopulation stabilized at 20% to 30% for the next 20 generations.

As a further confirmation of the cancer cell stem phenotype, qRT-PCR was carried out for the expression of Twist, CD44, and CD24 transcript levels in the CD44⁺/CD24^{-/low} and CD44⁺/CD24⁺ subpopulations. CD24 expression was four-fold lower (P=0.002) in the CD44⁺/CD24^{-/low} subpopulation compared with the CD44⁺/CD24⁺ subpopulation (FIG. 5B). In addition, Twist expression was 15% higher (P=0.003) in the CD44⁺/CD24^{-/low} subpopulation compared with the CD44⁺/CD24⁺ subpopulation. No significant difference was observed in the CD44 expression between these two subpopulations.

### Example 6

### Increased Mammosphere Formation in Twist-Overexpressing Cells

Because the ability to form mammospheres is a characteristic of cancer stem cells, the ability of MCF-10A/Twist and MCF-7/Twist cells to form mammospheres in culture was evaluated. MCF-7/Twist cells formed mammospheres that were disaggregated, and unlike the classic rounded mammosphere phenotype observed in culture (FIG. 5C). Similar results were reported using the highly aggressive breast cancer cell line, MDA-MB-231, which also exhibits a large subpopulation of CD44⁺/CD24^{-/low} cells. Nonetheless, these cells were alive and present in significantly higher numbers (55 vs 30, P<0.0001) compared with parental MCF-7 cells when analyzed by the Calcein-AM green staining (lower rows). With respect to MCF-10A/Twist cells, significantly larger numbers of mammospheres (53 vs 32, P=0.002) were generated compared with the parental MCF-10A cells (FIG. 5D). This was observed in two independent MCF-10A/Twist clones. In addition, the mammospheres generated using MCF-10A/Twist cells were significantly larger than the parental MCF-10A cells.

### Example 7

### Twist Downregulates CD24 Expression in Breast Cancer Cells

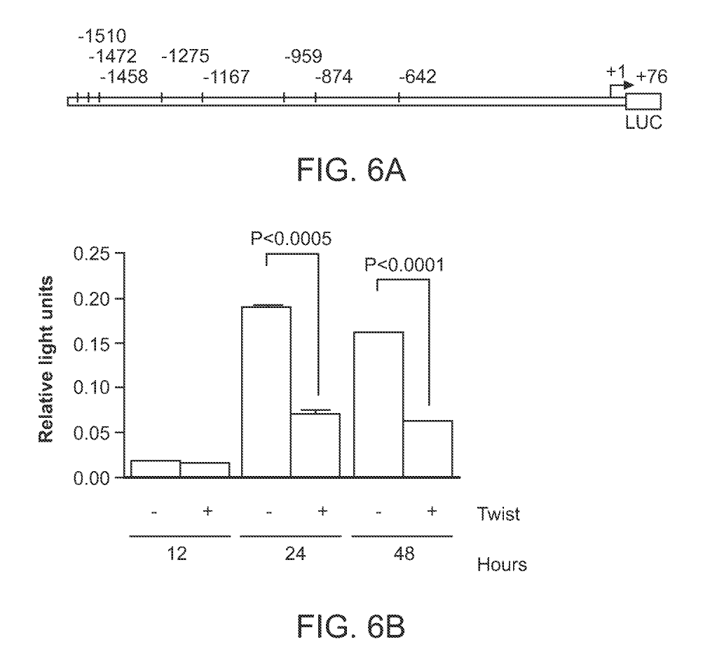

After the observation that Twist decreased CD24 expression, whether this repression was transcriptionally regulated was studied. The 1.6-kb CD24 promoter region was analyzed for E-box sequences (CANNTG) to which Twist can potentially bind. As shown in FIG. 6A, the putative CD24 promoter contains eight E-box sequences. This region was PCR-amplified and cloned into the pGL4 luciferase reporter plasmid. Transient transfection assays using the CD24 promoter-reporter construct and a Twist expression plasmid in MCF-7 cells showed a significant down-regulation of the reporter gene, 24 and 48 hours after transfection (FIG. 6B). A further confirmation of this regulation was demonstrated using protein extracts from MCF-7/Twist cells and scored for CD24 protein levels. As shown in FIG. 7, CD24 protein expression was significantly reduced in MCF-7/Twist compared with parental MCF-7 cells.

### Example 8

### Twist Binds In vivo to E-box Sequences in the CD24 Promoter

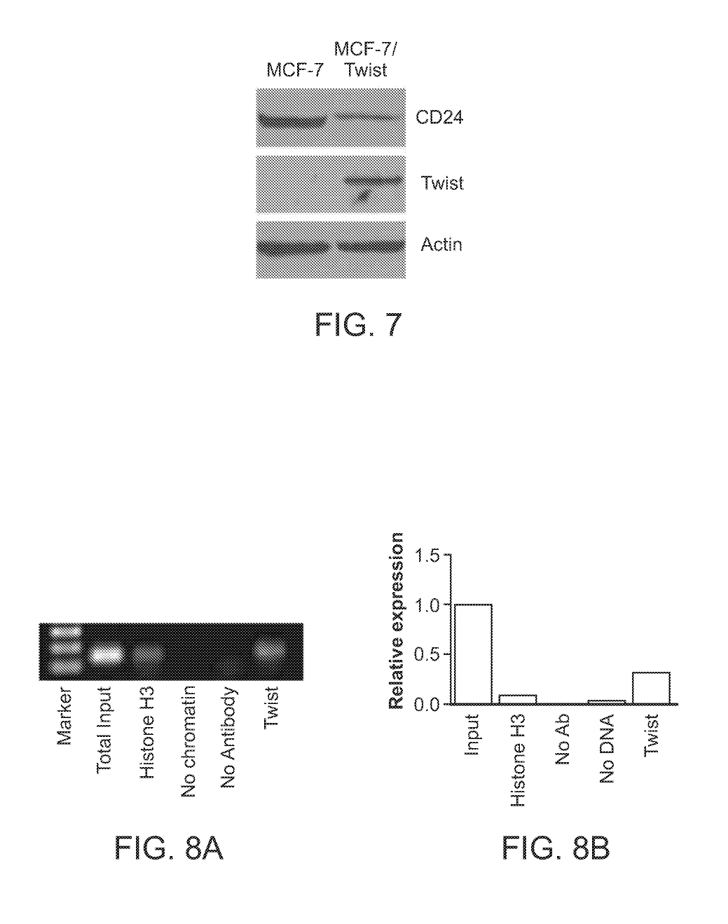

To establish that the down-regulation of CD24 by Twist was due to the direct binding of Twist to the CD24 promoter sequence, chromatin immunoprecipitation (ChIP) assays were performed using MCF-7 cells overexpressing Twist (FIG. 8A). A set of PCR primers (5′-TGCCCCTTAGAATTGCTGTT-3′ and 5′-TCATTGAACCTGGAAGTGG-3′) was designed for specific amplification of the Twist binding sites (E-boxes) from −1256 and −1107 within the CD24 promoter. As shown in FIG. 8A, the use of this primer set in PCR of ChIP DNA generated from Twist immunoprecipitations resulted in a specific amplified product of 149 bp, whereas no amplification was seen in the samples that were processed in the absence of precipitating antibody or no input chromatin. The PCR was confirmed by qRT-PCR amplification (FIG. 8B). These results indicate that Twist binds directly or as part of a complex to the endogenous CD24 promoter in vivo.

### Example 9

### Twist is Over-Expressed in Primary Breast Carcinomas

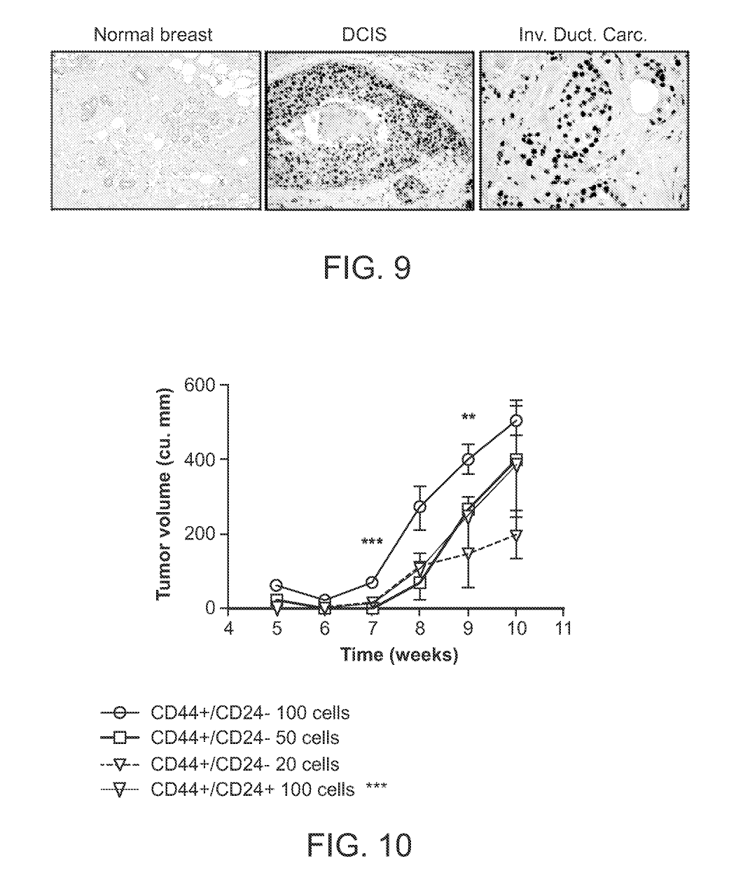

Twist expression and its functions have been primarily studied in mesodermal lineages. In contrast, its expression levels and functions within the epithelial environment have not been extensively studied. Twist protein expression and its functions in breast epithelium were evaluated by performing immunohistochemistry on normal breast tissue samples, ductal carcinoma in situ (DCIS) samples, and in primary breast carcinoma samples (Grade 2 and higher, FIG. 9). The results showed that high-grade breast tumors express Twist at elevated levels as compared to normal breast tissues. These data are in agreement with earlier findings, which showed that Twist mRNA and protein expression is elevated in invasive breast carcinoma. Both sets of data indicate a functional role for Twist in altering breast epithelium and in the pathogenesis of human breast cancer.

### Example 10

### The Twist⁺/CD44⁺/CD24^{-/low} Sub-Population Exhibits Increased Tumorigenic Potential In Vivo

A hallmark of cancer stem cells is the ability to form tumors with limited number of cells. In order to determine the minimum number of cells of the Twist⁺/CD44⁺/CD24^{-/low} sub-population required to initiate and establish tumor growth within the mammary fat pad, we injected 100, 50, and 20 purified cells into the orthotopic site in SCID mice, as well as 100 cells of the Twist⁺/CD44⁺/CD24⁺ purified sub-population. Following an incubation period of seven weeks, tumor growth was observed in mice injected with 20 cells (FIG. 10). There was a trend towards increased latency as the cell inoculums became smaller, with tumor uptake time being approximately two to three weeks quicker in the 100 cell inoculums of the Twist⁺/CD44⁺/CD24^{-/low} subpopulation. Moreover the Twist⁺/CD44⁺/CD24⁺ sub-population grew much slower than the Twist⁺/CD44⁺/CD24^{-/low} sub-population.

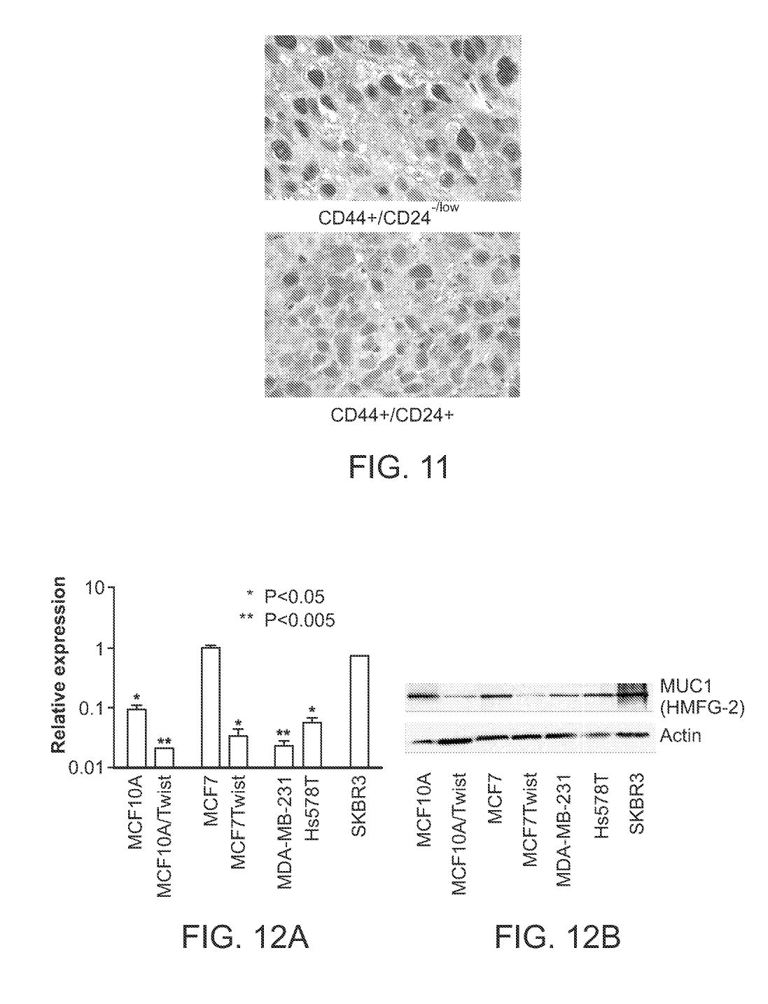

In order to study the expression levels of Twist in both the CD44⁺/CD24^{-/low} and the CD44⁺/CD24⁺ derived tumors, immunohistochemistry was performed on tumor sections. Tumors generated from the Twist⁺/CD44⁺/CD24⁺ sub-population had lower Twist expression as compared to tumors derived from the Twist⁺/CD44⁺/CD24^{-/low} sub-population (FIG. 11).

### Example 11

### Loss of MUC1 Expression in Twist Expressing Breast Cancer Cell Lines

MUC1 expression levels during mammary biogenesis have been associated with bipotent and myoepithelial progenitor cells. As Twist expression induces EMT, and as EMT cells and myoepithelial cells share a distinct lineage, MUC1 expression in MCF-7/Twist cells was analyzed. Microarray data indicated a 26 fold decrease of MUC1 expression. To verify this result, MUC1 expression was measured both by qRT-PCR and immunoblots in a panel of breast cancer cell lines including Twist-overexpressing, as well as Twist transgenic breast cancer cell lines. As shown in FIG. 12A, over-expression of Twist in both normal immortalized mammary epithelial cells, MCF 10A, as well as in MCF-7 cells, greatly reduced the expression of MUC1. In addition, MDA-MB-231 and Hs578T breast cancer cells, which have high endogenous Twist expression, exhibit relatively low MUC1 expression. On the contrary, the breast cancer cells with low endogenous Twist—MCF 10A, MCF7, and SKBR3—had relatively high levels of MUC1 expression. Importantly, the protein levels in the identical cell lines paralleled that of the transcript levels (FIG. 12B). These results strongly support a function for Twist in down-regulating MUC1, which contributes to the generation of a myoepithelial phenotype.

### Example 12

### Cell Surface Expression of MUC1 on Twist Expressing Breast Cancer Cells

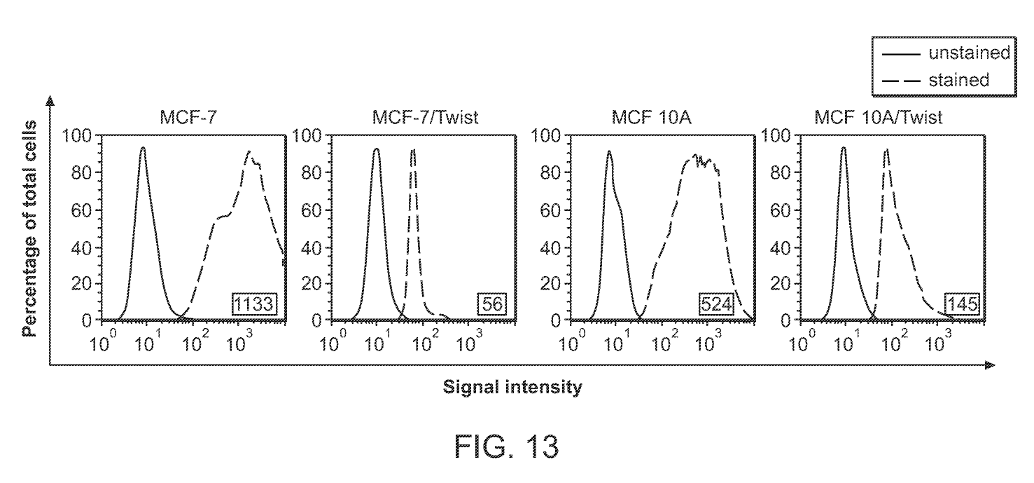

One of the characteristics of myoepithelial progenitor cells is the loss of the MUC1 expression on the cell surface. As preliminary data clearly indicated that MUC1 levels were decreased by Twist expression as assayed by both qRT-PCR and immunoblot, MUC1 expression on the cell surface of two different transgenic cells, MCF 10A/Twist and MCF-7/Twist, was analyzed using the FITC conjugated MUC1 antibody (BD Biosciences). This antibody recognizes the tandem repeats of the extracellular domain. As shown in FIG. 13, expression of Twist resulted in a decrease of the mean signal intensities of MUC1 by approximately 20 fold in MCF-7 cells. A similar trend was also observed in MCF 10A/Twist cells (FIG. 13). Decreased signal intensities correspond to lower MUC1 expression on the cell surface. These data strongly support the hypothesis that Twist expression promotes cancer initiating cells by altering the expression of specific gene signatures.

### Example 13

### Twist Down-Regulates p27 Expression in Breast Cancer Cells

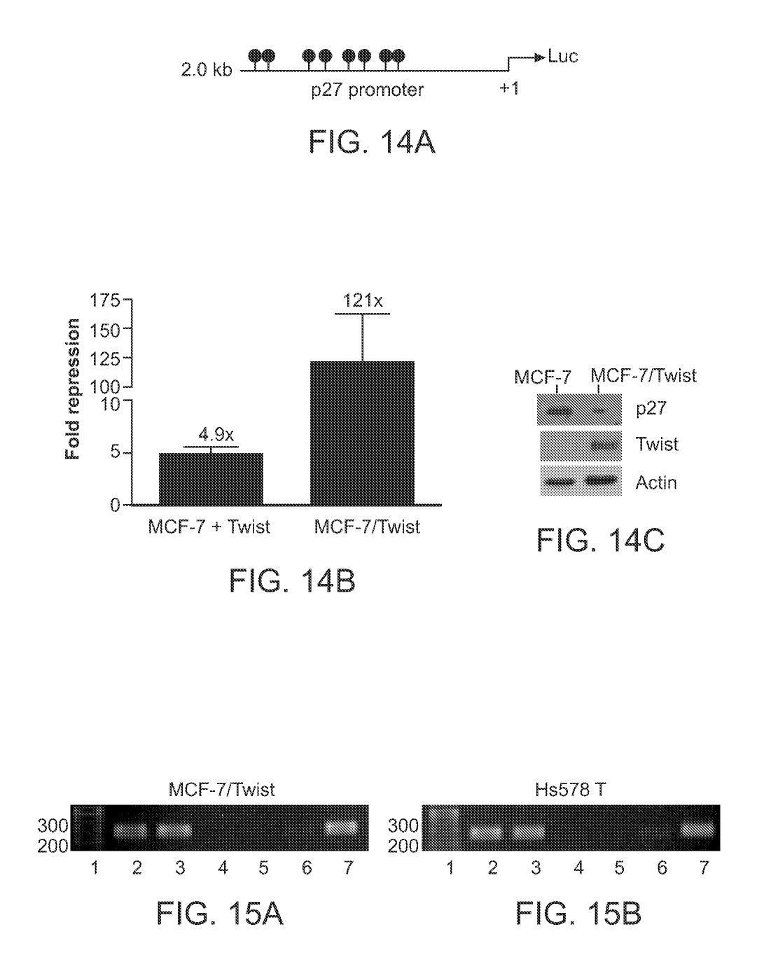

Following the observation that Twist induces an epithelial-mesenchymal transition and metastasis, it became important to identify the down-stream target genes of Twist. Based on the available information from the literature, it was apparent that very few target genes of Twist have been characterized. Affymetrix microarray analysis was performed using RNA from MCF-7 vector control and MCF-7/Twist cells. One of the genes that was identified as being significantly repressed was p27. As Twist binds to E-box sequence (CANNTG), the putative p27 promoter region was searched for this motif. As shown in the FIG. 14A, the putative 2.0 Kb promoter region contains E-box sequences. Transient transfection assays using the p27 promoter-reporter construct in both MCF-7 (exogenous Twist expression plasmid added) and in MCF-7/Twist cells clearly showed a down-regulation of the reporter gene (FIG. 14B). This regulation was further verified using protein extracts from MCF-7/Twist cells and examining for p27 protein levels. As shown in FIG. 14C, in MCF-7/Twist cells, the p27 protein was significantly reduced as compared to MCF-7. The importance of this finding is that loss of p27 in breast cancer patients can be an indicator of poor prognosis.

### Example 14

### In Vivo Binding of Twist to its Cognate Sequence in p27 Promoter

As Twist over-expression in MCF-7 cells resulted in the down-regulation of p27, it was of interest to determine whether this effect was due to the direct binding of Twist within the p27 promoter sequence. To address this, chromatin immunoprecipitation (ChIP) assays were carried out using MCF-7/Twist. In addition, ChIP assays using Hs578 T, a breast cancer cell line that has a high level of endogenous Twist, were also performed. A set of primers was designed for specific amplification of the Twist binding sites (E-boxes) from −2078 and −1838 bp within the p27 promoter. As shown in FIGS. 15A and 15B (lane 7), PCR of ChIP DNA generated from anti-Twist immunoprecipitations resulted in amplified products of 241 base pairs. The same amplification products were seen in positive control experiments from unprocessed chromatin as well as from the ChIP DNA generated from anti-acetyl-Histone H3 precipitations (lanes 2 and 3 respectively). In contrast, non-specific antibody mediated precipitations resulted in DNA templates that were not amplified (lane 4). These results indicate that Twist binds either directly, or as part of a complex, to the endogenous p27 promoter in vivo.

### Example 15

### Twist Expression Induces Breast Cancer Metastasis to the Bones and Lungs

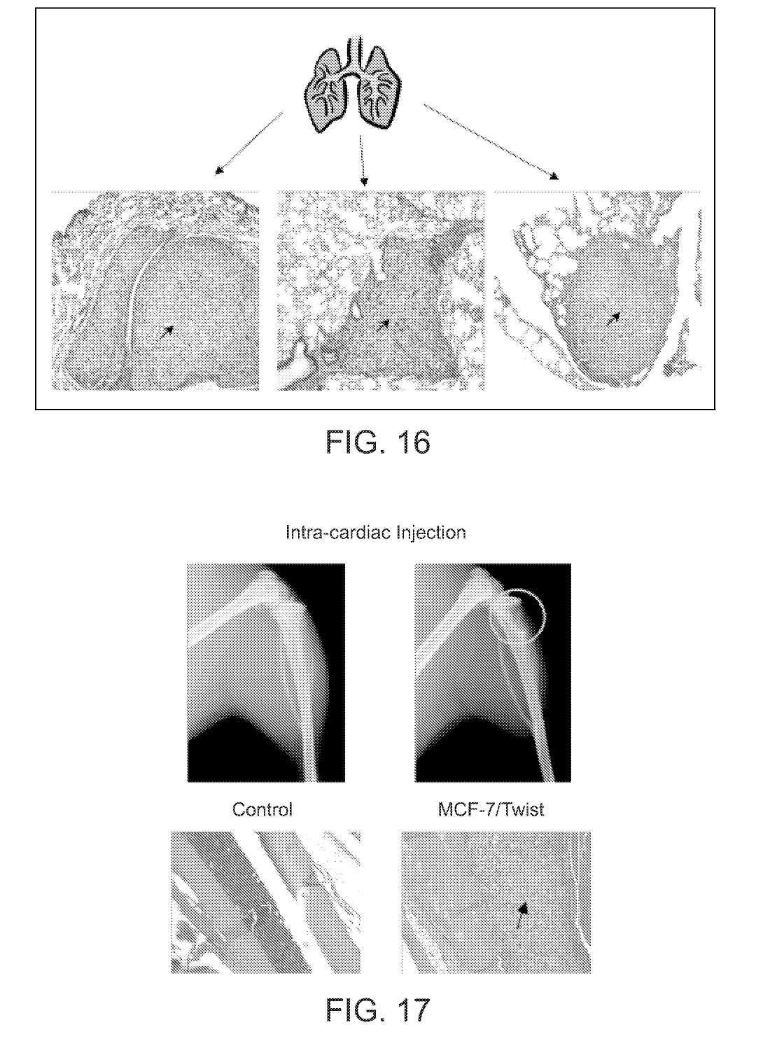

Metastases are the single largest cause of death from cancer. The cellular transformations that modulate metastasis are analogous to some of the cellular changes that are required for normal embryonic development including epithelial-mesenchymal transition (EMT). Since Twist overexpression induces EMT, it became important to determine whether Twist overexpression was sufficient to promote metastasis. Two months following orthotopic injections (n=15) and intra-cardiac injections (n=5) the animals were X-rayed using a Faxitron instrument, sacrificed, and analyzed for distinct, visible metastasis. All the animals injected with MCF-7/Twist cells exhibited lung (FIG. 16) and bone metastasis (FIG. 17). However, no metastatic lesions were observed in the animals with the parental MCF-7 cell derived xenografts. This result clearly demonstrates that over-expression of Twist in breast epithelium is able to promote intravasation, extravasation and establishment in a visceral organ like the bone and lung. Moreover, proliferation of MCF-7/Twist cells in the bone leads to an osteoclastic phenotype.

### Example 16

### Twist Up-Regulates Choline Kinase Expression in MCF-7 Cells

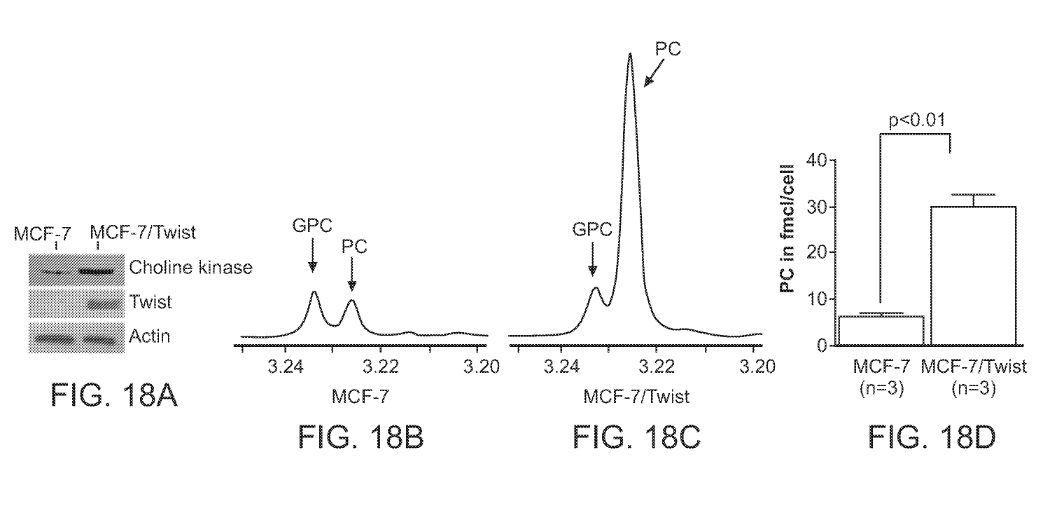

Over-expression of choline kinase and its effects on choline metabolism (phosphocholine and glycerophosphocholine) has been demonstrated as a useful marker in identifying invasive phenotypes. Based on the genotypic alterations observed in MCF-7/Twist cells, the Affymetrix data was analyzed for the expression of choline kinase in MCF-7/Twist cells. Interestingly, a two-fold increase in choline kinase mRNA levels was observed in MCF-7/Twist cells when compared to the parental MCF-7 cells. Subsequent analysis by immunoblot clearly showed that the choline kinase protein levels were three to four fold more in MCF-7/Twist than in MCF-7 cells (FIG. 18A). In addition, MCF-7/Twist cells showed a significant increase in the expression of phosphocholine levels, an indicator of choline kinase activity (FIGS. 18B-18D). Overall, the results indicate that the over-expression of Twist can increase choline kinase levels and activity.

### Example 17

### Increased Phe and Lactate in Twist Over-Expressing MCF-7 Cells

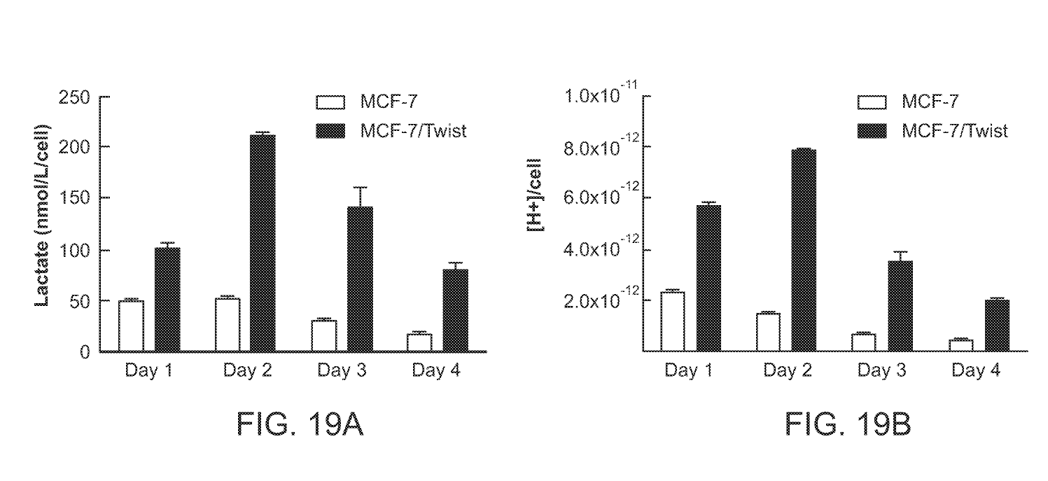

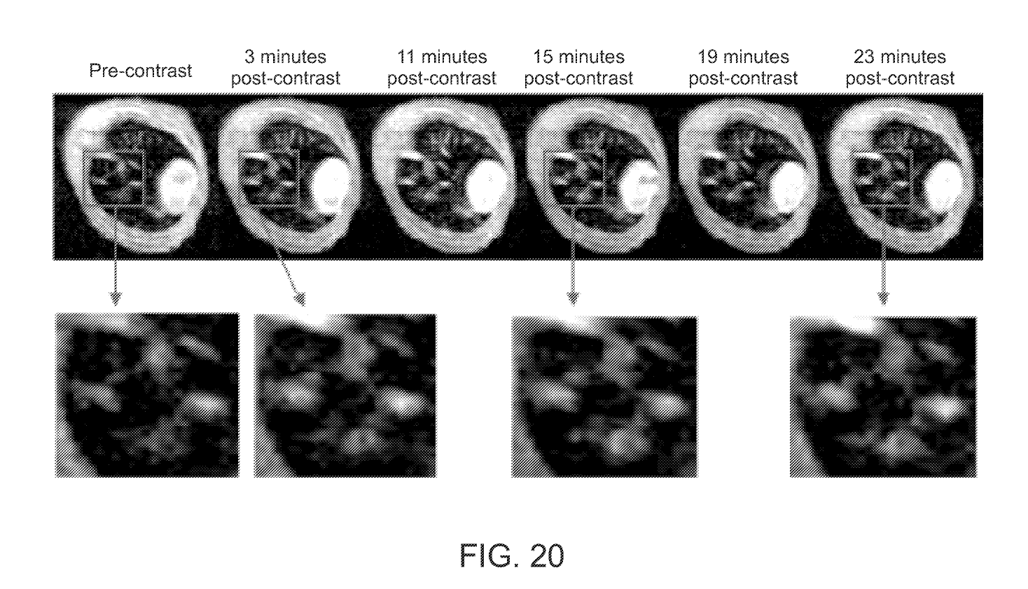

Cancer cells exhibit an increased production of lactate and an acidic extracellular milieu. These conditions have been demonstrated to favor tumor growth, invasion, and development. As MCF-7/Twist cells are highly aggressive, it was important to determine whether the lactate and pHe were altered in vitro relative to MCF-7 cells. Initial microarray analysis provided strong evidence of up-regulated lactate dehydrogenase levels in MCF-7/Twist cells (250 fold) as compared to the parental MCF-7 cells. To verify whether or not this corresponded to increased extracellular lactate and hydrogen ions [H⁺] levels, their levels in MCF-7 and MCF-7/Twist medium were measured over a period of four days. As depicted in FIGS. 19A and 19B, both lactate and [H⁺] ions increased by approximately 4 fold (day 2) in MCF-7/Twist medium as compared to MCF-7. Without being bound by any particular theory, it is believed that the resulting acidic extracellular pH promotes aggressive behavior by altering proteolytic activity and/or remodeling the extracellular matrix (ECM).

### Example 18

### Non-Invasive Imaging of Breast Cancer Metastasis

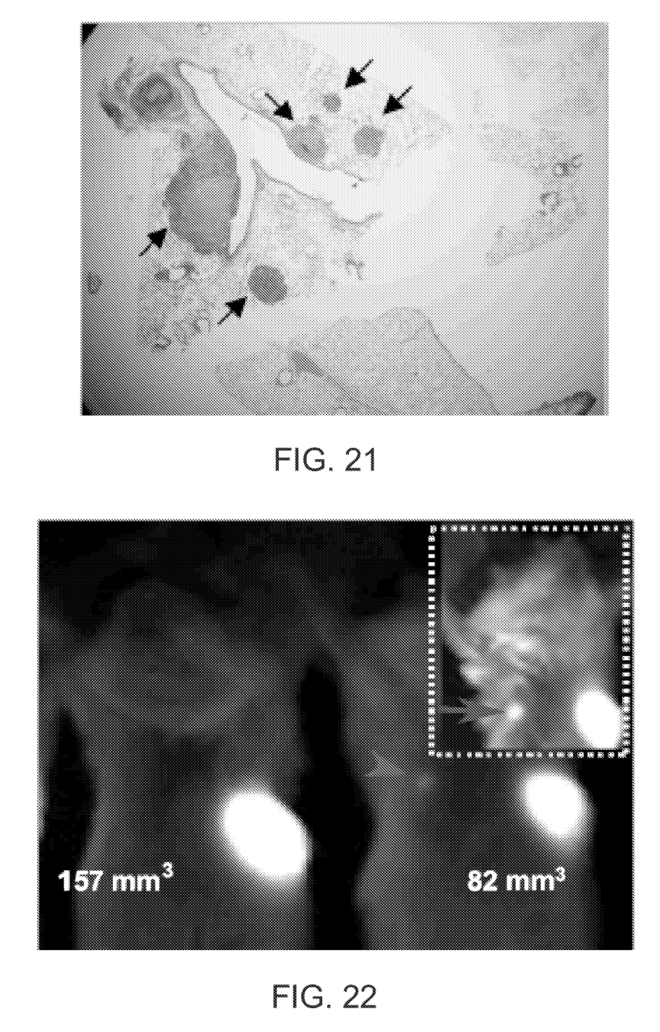

An important criterion for studying oncogenic functions is the detection of metastatic nodules using noninvasive techniques. The utility of MRI to detect breast cancer to lung metastasis (FIG. 20), which has been directly confirmed by histopathology (FIG. 21), has been demonstrated. Also, with respect to detecting metastasis using optical imaging, the use of a red fluorescence protein (tdTomato) for non-invasive optical tracking of cancer cells in a metastatic model of breast cancer has been developed and characterized. Using this system, metastatic progression from the mammary fat pad to the contra-lateral mammary fat, lymph nodes, and lungs of live animals was demonstrated without surgical intervention (FIG. 22).

### Example 19

### Estrogen Receptor (ER) is Down-Regulated in Twist Over-Expressing Cancer Cell Lines

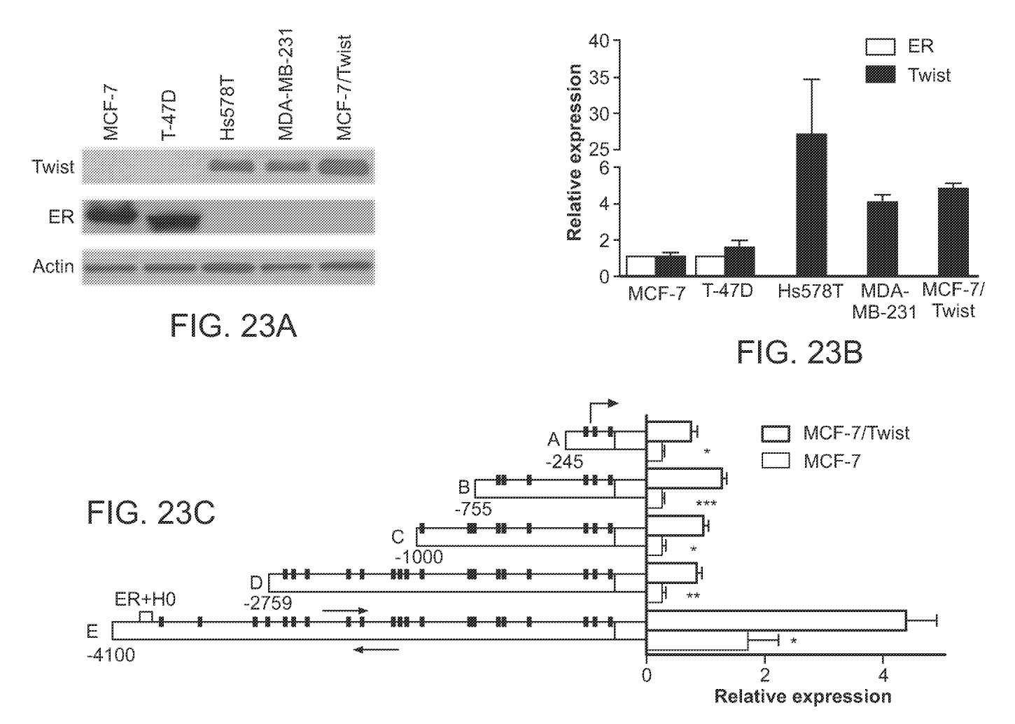

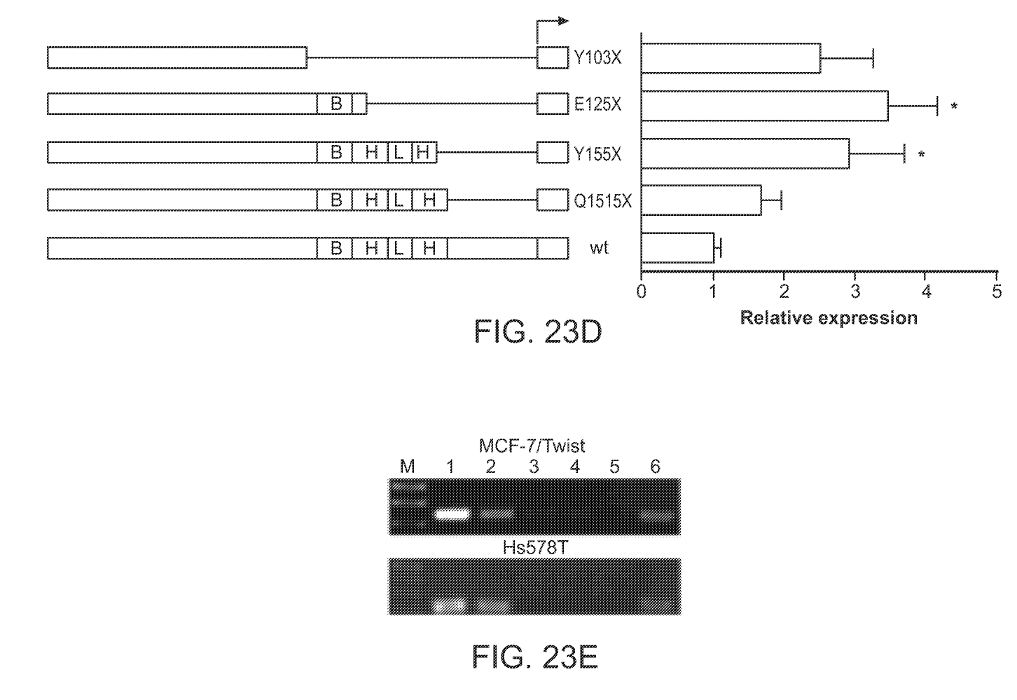

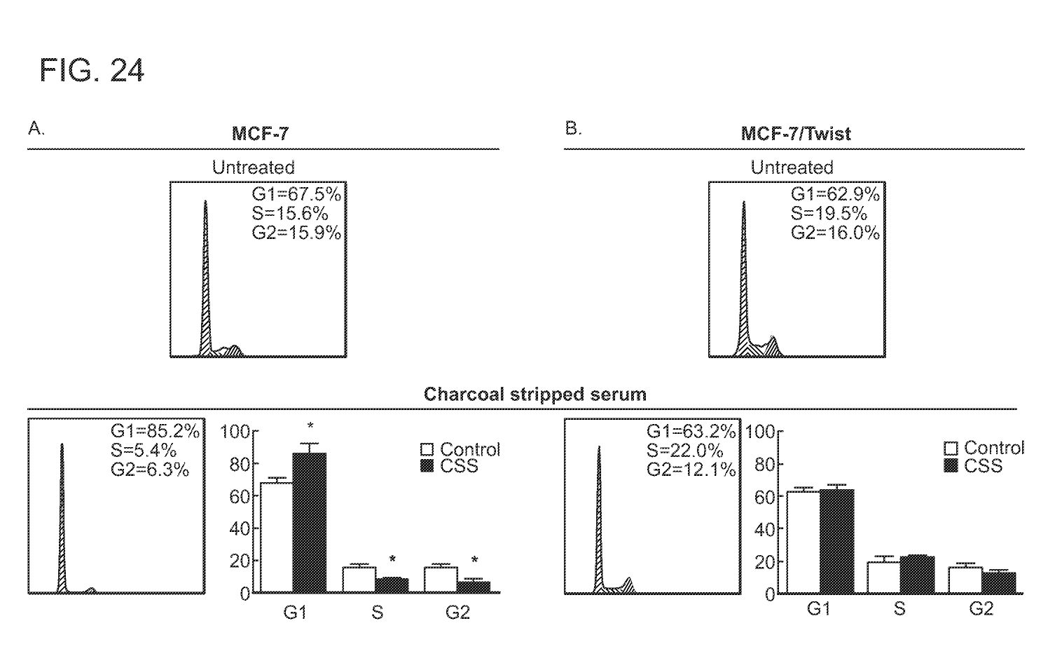

Analysis of the Affymetrix microarray for differential gene expression within the MCF-7/Twist cell line also identified the ER transcript, which was down-regulated by 13 fold. To confirm this finding, breast cancer cell lines were evaluated for Twist and ER expression by immunoblotting and qRT-PCR. As shown in FIGS. 23A and 23B, there was an inverse correlation between Twist and ER protein and mRNA transcript levels within the cell lines tested.

### Example 20

### Twist Represses ER Promoter Activity in Breast Cancer Cells

To functionally confirm the regulatory role of Twist in ER down-regulation, promoter-reporter assays were carried out in breast cancer cell lines. The 4 kb ER promoter has 26 canonical Ebox sequences (CANNTG) to which Twist can potentially bind (FIG. 23C). Promoter reporter assays were carried out in MCF-7 (FIG. 23C) and MCF-7/Twist cells (data not shown). Twist repressed the full-length ER promoter by 2.5 fold, while the other deletion constructs were repressed from 2.5 to 3 fold.

In order to confirm the role of the bHLH regions of Twist in binding the ER promoter, the full-length ER promoter and Twist bHLH deletion mutants were used to assay for ER promoter repression. As seen in FIG. 23D, none of the Twist mutants demonstrated repression comparable to wild-type Twist, except for the deletion mutant Q161X, which was downstream of the bHLH domain.

### Example 21

### Twist Binds Directly to E-Boxes within the ER Promoter

To determine whether Twist binds directly to the ER promoter, ChIP assays using MCF-7/Twist and Hs578T cell lines were carried out (FIG. 23E). Hs578T is an ER negative breast cancer cell line with high levels of endogenous Twist. PCR from Twist antibody immunoprecipitations resulted in a specific amplified product of 241 base pairs. The identical amplification product was seen in the positive controls from both unprocessed chromatin and acetyl histone H3 precipitations. Non-specific antibody mediated ChIP resulted in negligible amplification. In addition, no amplification was seen in samples that were processed without antibody or chromatin. These results indicated that Twist binds directly or as part of a complex to the endogenous ER promoter.

### Example 22

### Twist Causes Hormone Independence in Breast Cells

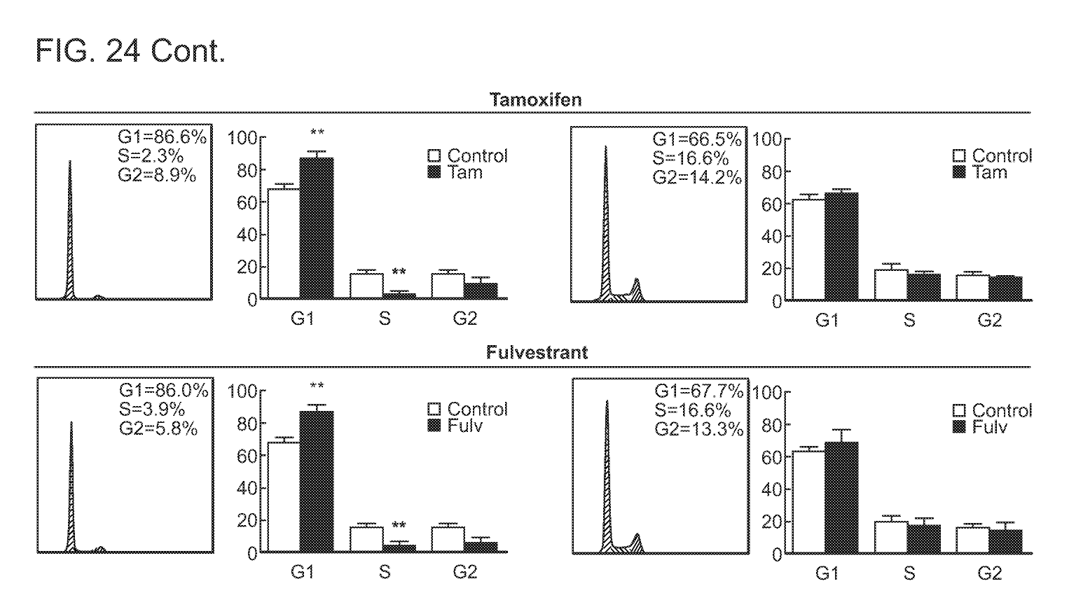

As increased Twist expression was observed in ER negative cell lines, it seemed likely that Twist promoted hormone independence by repressing ER expression. To confirm this observation, MCF-7 (ER positive) and MCF-7/Twist (ER negative) cells were grown for three days in estrogen depleted media containing 5% charcoal stripped serum (CSS) and cell cycle distribution was analyzed by flow cytometry (FIG. 24). Proliferation of MCF-7 cells (FIG. 24A) was significantly reduced in CSS (S=5.4%) compared to untreated cells (S=15.6%, P<0.05), but not in MCF-7/Twist cells (S=22.0% vs. 19.5%, P>0.05) as shown in FIG. 24B. Moreover, the percentages of cells in all three phases of the cell cycle was significantly different between MCF-7 and MCF-7/Twist cells treated with CSS—G1=85.2% vs. 63.2%, P<0.05; S=5.4% vs. 22.0%, P<0.005; and G2/M=6.3% vs. 12.1%, P<0.05. The difference was insignificant in untreated controls of both MCF-7 and MCF-7/Twist. These results support the earlier data indicating that the down-regulation of ER by Twist in MCF-7 cells leads to estrogen independent growth.

### Example 23

### Twist Promotes Hormone Resistance in Breast Cancer Cells

To investigate if the loss of ER brought about by Twist caused hormone resistance in breast cells, MCF-7 and MCF-7/Twist cells were treated with the selective estrogen receptor modulator (SERM) tamoxifen, and the selective estrogen receptor down-regulator (SERD) fulvestrant. As seen in FIG. 24A, MCF-7 cells were significantly arrested in the presence of tamoxifen compared to untreated cells (S=2.3% vs. 15.6%, P<0.005). On the other hand, FIG. 24B shows that MCF-7/Twist cells were largely unaffected by tamoxifen treatment (S=16.6% vs. 19.5%, P>0.05). Also, the G1 and S phases of the cell cycle were significantly altered in MCF-7 and MCF-7/Twist cells treated with tamoxifen (G1=86.6% vs. 66.5%, P<0.005; S=2.3% vs. 16.6%, P<0.005; G2/M=8.9% vs. 14.2%, P>0.05). Incubation with fulvestrant exhibited results comparable to those with tamoxifen (FIGS. 24A and 24B). The S-phase of MCF-7 cells was significantly repressed by fulvestrant compared to untreated controls (S=3.9% vs. 15.6%, P<0.005), while MCF-7/Twist cells were unaffected by the treatment (S=16.6% vs. 19.5%, P>0.05). Similarly, all cell cycle phases of MCF-7 cells were affected by fulvestrant compared to MCF-7/Twist cells (G1=86% vs. 67.7%, P<0.005; S=3.9% vs. 16.6%, P<0.0005; G2/M=5.8% vs. 13.3%, P<0.05). As shown previously, differences in untreated cells were not significant.

### Example 24

### Twist Promotes Growth of Breast Tumors in the Absence of Estrogen

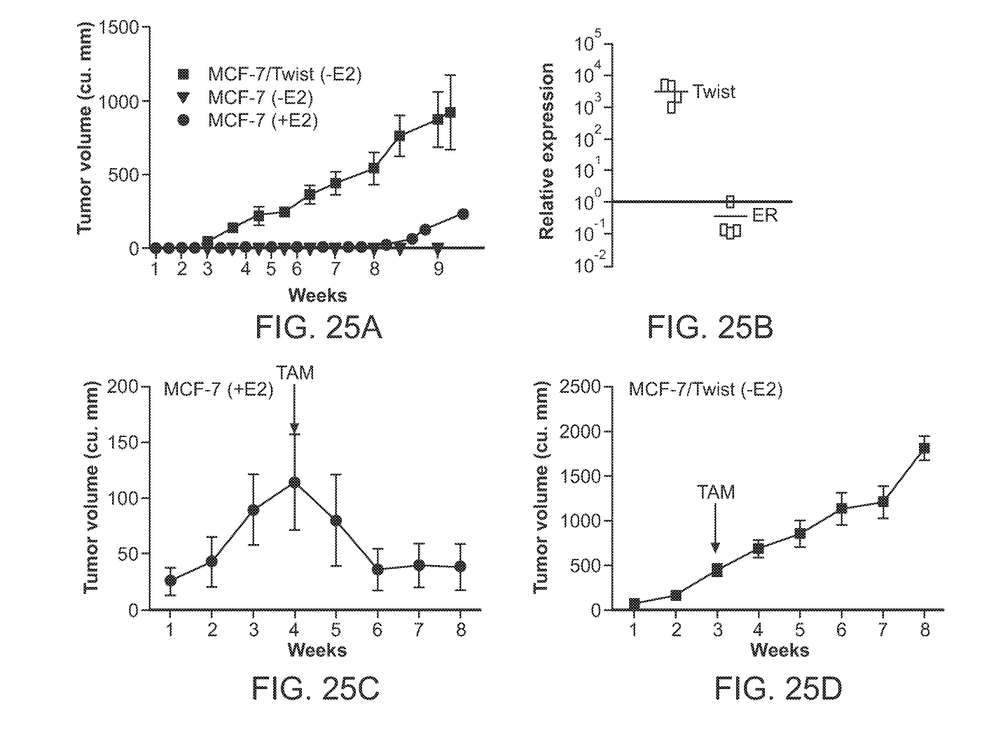

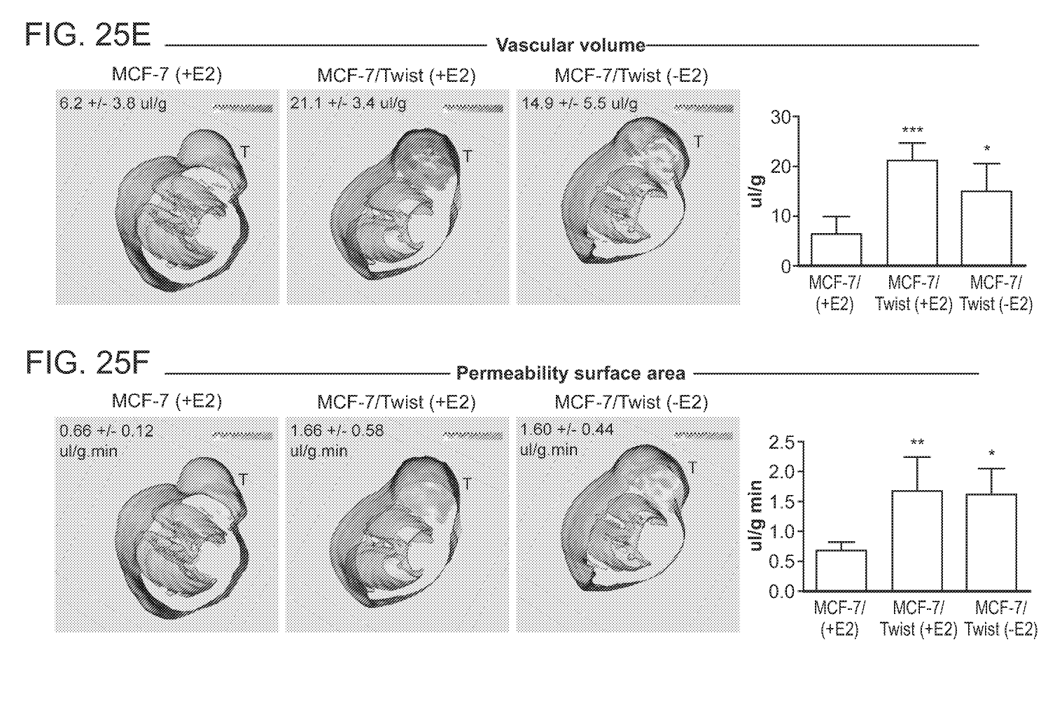

To further demonstrate that over-expression of Twist induced estrogen independence in vivo, MCF-7/Twist cells were injected orthotopically into the mammary fat pads of SCID mice, which were not supplemented with estrogen. As seen in FIG. 25A, MCF-7/Twist xenografts produced large tumors (greater >250 mm3) within four to five weeks of incubation. These results confirmed that MCF-7/Twist cells are estrogen independent in vivo. In order to confirm that the expression of Twist and ER in tumors was similar to that of MCF-7/Twist cells, RNA from four tumors was isolated and analyzed by qRT-PCR using Twist and ER specific primers. As shown in FIG. 25B, expression of Twist was inversely correlated with levels of ER transcripts.

Next, mice (n=10) were injected with MCF-7/Twist and MCF-7 cells in the presence of estrogen (17β-estradiol pellet implanted in the back). After 3-4 weeks of growth, all mice were implanted with a tamoxifen pellet. As seen in FIGS. 25C and 25D, MCF-7/Twist tumors were unaffected by tamoxifen, while MCF-7 tumors regressed to pre-treatment levels.

### Example 25

### Twist Increases Vascular Volume and Vascular Permeability of Breast Tumors in Mice

Functional magnetic resonance imaging (fMRI) was used to non-invasively analyze the vascular volume (VV) and PS area product values in vivo. FIGS. 25E and 25F display representative false color-coded MRI generated 3-D transverse slices of xenograft tumors using MCF-7 and MCF-7/Twist cells in mice. The average tumor VV in MCF-7 (+E2) and MCF-7/Twist (−E2) xenografts was 6.2 and 14.9 μl/g, respectively (FIG. 25E). The average tumor PS in MCF-7 (+E2) and MCF-7/Twist (−E2) xenografts was 0.66 and 1.60 μl/g·min, respectively (FIG. 25F). Both results were significant according to the Scheffe test (F=15.9 and 7.04, respectively). VV and PS values in MCF-7 vector control xenografts were comparable to those in MCF-7 xenografts (data not shown), and were consistent with the previous report. VV and PS in MCF-7/Twist (−E2) xenografts were 21.1 ul/g and 1.66 ul/g.min, respectively. These values were significantly higher than those in MCF-7 (+E2) controls (F=5.48 and 6.23 respectively). There was no significant difference between estrogen supplemented and non-supplemented MCF-7/Twist xenografts for VV and PS (F=3.00 and 0.03, respectively).

### Example 26

### Twist Knock Down Causes Re-Expression of ER

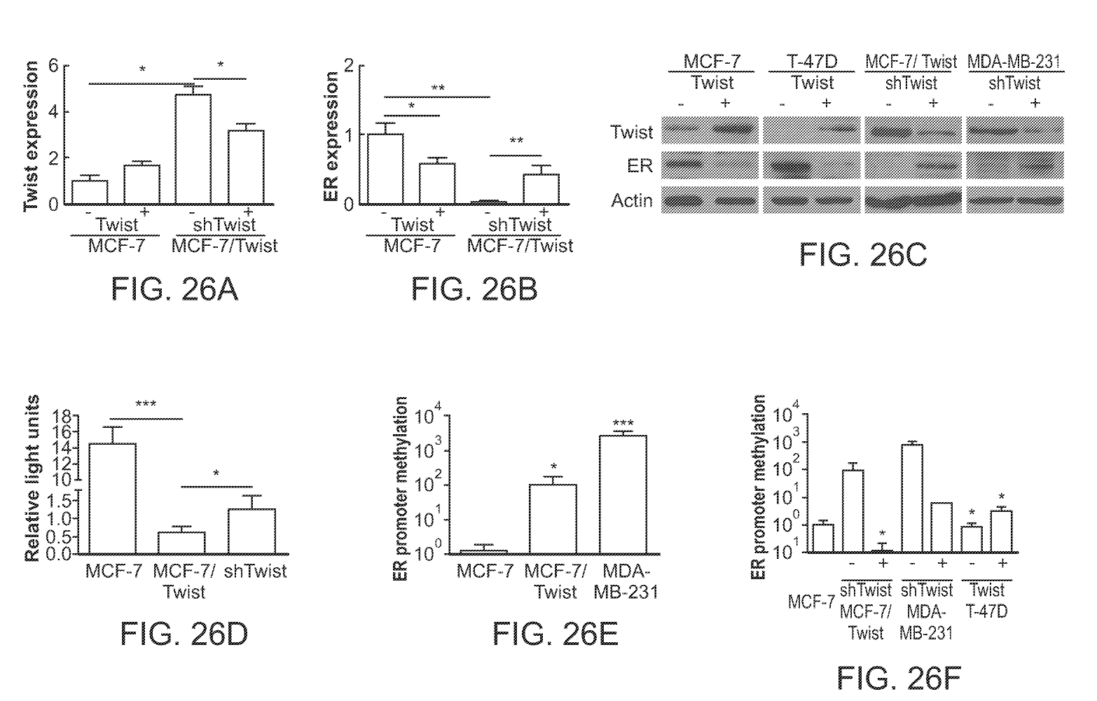

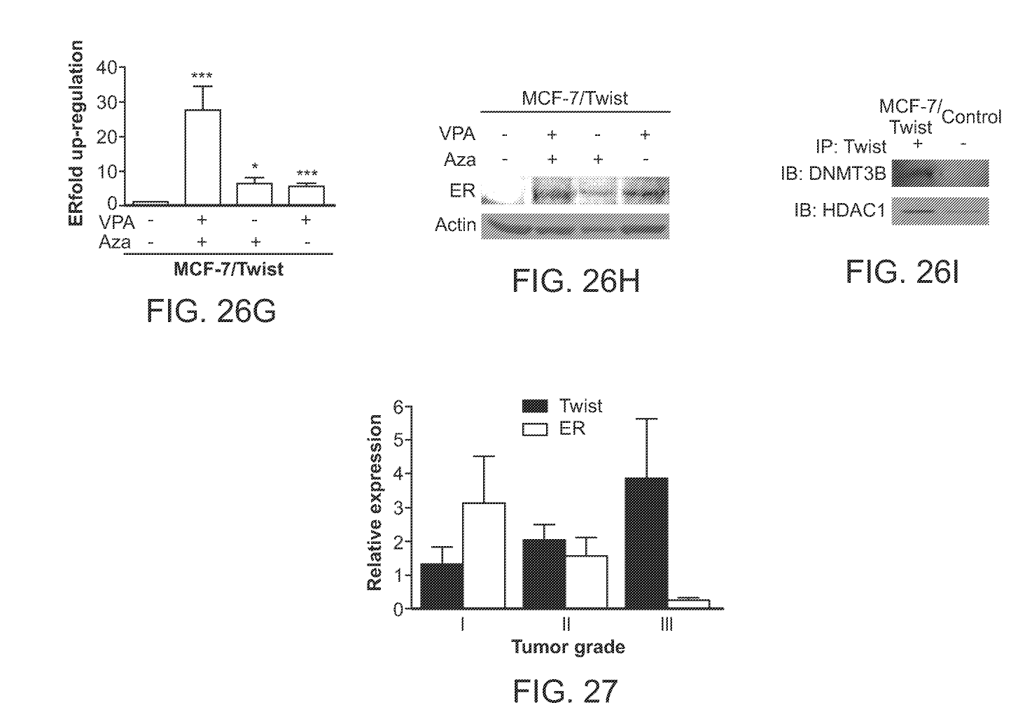

To further investigate the regulation of ER by Twist, MCF-7/Twist cells with Twist knock-downs and MCF-7 cells with Twist knock-ups were generated. As seen in FIGS. 26A and 26B, knock down of Twist in MCF-7/Twist cells caused a significant drop in mRNA levels of Twist accompanied by an increase in ER protein levels (FIG. 26C). Transient expression of Twist, on the other hand, caused a significant drop in ER transcript in MCF-7 cells and a similar change in protein levels. It was also demonstrated that Her-2/neu protein levels were low in MCF-7/Twist cells, which indicates that the effect of Twist on ER is not mediated by Her-2/neu (data not shown). Furthermore, it was determined that the reactivation of ER in the Twist knock-down clones was functionally active. For this purpose, the ERE-luciferase construct was used as a functional reporter system for the in vitro studies. As seen in FIG. 26D, MCF-7/Twist cells show a significant drop in the activation of the reporter indicating the lack of ER functionality in these cells. Importantly, the re-expression of ER by down-regulating Twist in MCF-7/Twist cells increased reporter activity, an indication of functional ER proteins.

### Example 27

### Twist Induces Hyper-Methylation of the ER Promoter

Promoter hypermethylation is a common mechanism of ER gene silencing and occurs in 5-49% of patient samples. Consistent with this, a significant increase in ER promoter methylation in MCF-7/Twist cells was observed by MS-qPCR analysis (FIG. 26E). Subsequently, transient Twist knock-down and knock-ups were used to validate our earlier observations. As seen in FIG. 26F, Twist over-expression in T-47D cells caused an increase in ER promoter methylation. On the other hand, Twist knock-down in MCF-7/Twist and MDA-MB-231 caused a significant decrease in ER promoter methylation.

In order to reverse the Twist induced methylation of the ER promoter, MCF-7/Twist cells were treated with the demethylating agent AZA, which resulted in a significant increase in ER transcript and protein levels as seen by qRT-PCR and immunoblotting (FIGS. 26G and 26H). To examine the mechanism underlying the increased methylation of ER brought about by Twist, the recruitment of methyltransferases to the ER gene was assessed. The de novo methyltransferase DNMT3B was co-immunoprecipitated by Twist from MCF-7/Twist lysates (FIG. 26I). Other methyltransferases such as DNMT1 and DNMT3A were not co-immunoprecipitated by Twist.

### Example 28

### Twist Promotes Histone Deacetylation of the ER Promoter

Regulation of genes via methylation is accompanied, in some cases, by an increase in histone deacetylase (HDAC) activity. In order to functionally study the role of HDACs in the regulation of ER, MCF-7/Twist cells were treated with the HDAC inhibitor valproic acid (VPA). As seen in FIGS. 26G and 26H, there was a significant increase in ER expression in these cells when treated with the inhibitor. Combined use of 5-aza 2′-deoxycytidine (AZA) and VPA was able to rescue ER to a higher degree then either inhibitor alone. Without being bound by any particular theory, it is believed that Twist recruited HDAC1 to the ER promoter, which led to deacetylation, and that this was correlated with lowered expression of ER (FIG. 26I).

### Example 29

### Twist and ER are Inversely Correlated in Breast Cancer Patients

To confirm the inverse correlation between Twist and ER expression seen in breast cancer cell lines, Twist and ER mRNA levels in human breast tumors were quantified by qRT-PCR. A total of 31 primary breast cancers (grade I=6, grade II=12, grade III=13) and four normal breast samples were analyzed (FIG. 27). An increase in Twist expression levels was observed with increasing tumor grade, while ER expression levels showed a concomitant decrease as tumor grade increased. The correlation in normalized (to normal breast tissue) expression between TWIST and ER genes, overall, and within each grade, was examined using the nonparametric, Spearman rank test. With the exception of Grade 3, an inverse relation was observed, with increasing Twist expression associated with decreasing ER expression, though not statistically significant. Among grade 3 tumors, increasing ER expression was associated with increasing Twist expression, though not significant. Overall, these results are consistent with the hypothesis that Twist downregulates ER in human breast cancers.

### Example 30

### Models for ER Regulation by Twist

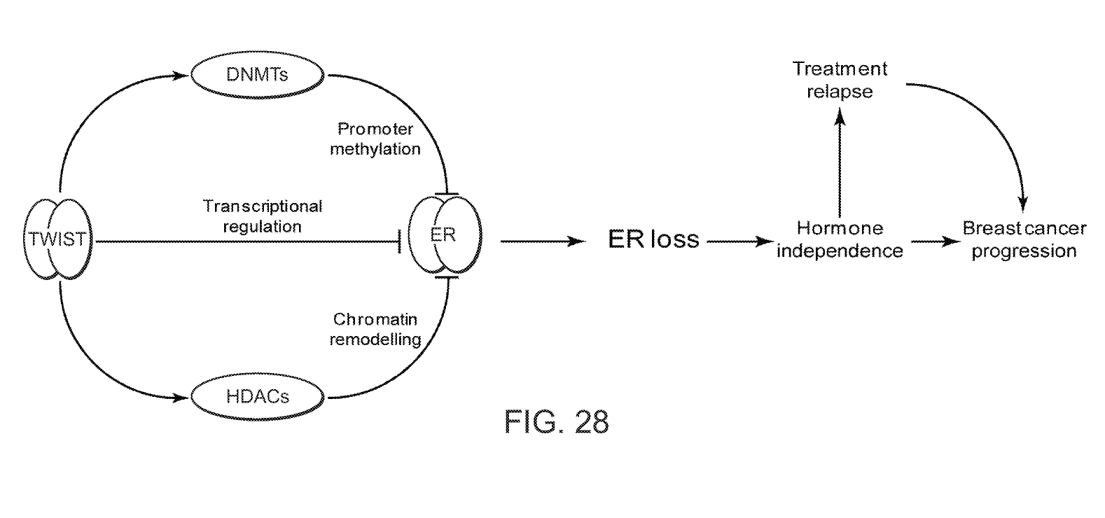

Without being bound by any particular theory, over-expression of Twist provides a mechanistic link between the development of aggressive breast cancer and the loss of ER expression, and offers a means to elucidate the ontogeny of ER negative hormone resistant breast cancers. Overall, the demonstrated alternative mechanistic explanations for the loss of ER expression in breast tumors, include: transcriptional regulation, promoter methylation, and chromatin remodeling. FIG. 28 shows a model for the regulation of ER by Twist in which Twist up-regulation causes the down-regulation of ER by direct transcriptional action. This regulation can be epigenetically enhanced by methylation and de-acetylation of the ER promoter. The loss of ER leads to hormone independence and resistance to targeted anti-estrogen therapy, which in turn leads to disease progression. It can be possible to reverse the down-regulation of ER by treating the MCF-7/Twist cells with a combination of Twist shRNA, methylation inhibitors and HDAC inhibitors leading to a rescue of ER expression and reversing the ER negative phenotype.

The results described herein above were obtained using the following methods and materials.

### Cell Culture

Cell lines were obtained from ATCC (Manassas, Va.) and maintained as instructed. Construction of MCF-7/Twist and MCF 10A/Twist cell lines have been described earlier (19, 23). For transient expression, cells were transfected using either retroviral constructs (generated in-house) or lentiviral constructs (Open Biosystems, Huntsville, Ala.). For hormone studies, cells were grown for two days in phenol-red free minimal essential media (PRF-MEM) containing 5% charcoal stripped serum (CSS). For anti-estrogen treatment, PRFMEM/5% CSS media was supplemented with 1 μM 4-hydroxytamoxifen (tamoxifen) or 1 nM fulvestrant. Three days following treatment, the cells were fixed in 70% ethanol and cell cycle analysis performed on a FacScan I (BD Biosciences, San Jose, Calif.). Independent experiments were repeated four times. Data was analyzed using FlowJo (Tree Star Inc., Ashland, Oreg.) and ModFit LT 2.0 software (Verity House Software, Topsham, Me.).

### Flow Cytometry

Cells were washed in Hank's balanced salt solution (HBSS) and harvested using 0.25% trypsin-EDTA (Invitrogen, Carlsbad, Calif.), counted, and resuspended in 1% fetal calf serum (FCS)-phosphate-buffered saline (PBS; 1×10⁶ cells/400 μl). Cells were incubated with CD44-fluorescein isothiocyanate (FITC) and CD24-phycoerythrin (PE; BD Pharmingen, San Diego, Calif.), along with respective controls for 45 minutes at 4° C. in the dark. After incubation, cells were centrifuged and resuspended in 500 pl of 1% FCS-PBS and acquired on a FACScan II Cytometer (BD Immunocytometry Systems, San Jose, Calif.). No-antibody and single-antibody controls were used to compensate the sample readings and for designating quadrants. Analysis was done using Cell Quest software (BD Immunocytometry Systems) on a Macintosh platform.

### Efflux Assays

Semiconfluent cells were washed once with HBSS and resuspended in growth medium at 1×10⁶ cells/ml and 5 μg/ml Hoechst 33342 (Sigma-Aldrich, St. Louis, Mo.) and incubated for 45 minutes at 37° C. in a constant-temperature water bath. Subsequently, the cells were washed once in cold HBSS/2% BSA and resuspended in warm medium and incubated for 45 minutes at 37° C. for efflux. Cells were centrifuged and mounted on a slide before photomicroscopy.

For Rhodamine 123 (Sigma-Aldrich) staining, cells were trypsinized and washed twice in PBS/1% FCS. One million cells were resuspended in 100 μl of PBS/1% FCS and stained with 100 to 500 ng/ml Rhodamine 123 for 15 to 30 minutes at 37° C. Cells were washed twice at room temperature, resuspended, and incubated without Rhodamine 123 for 15 to 30 minutes at 37° C. to allow for efflux to occur. Rhodamine efflux was subsequently determined by flow cytometry on a FACScan II flow cytometer.

### ALDEFLUOR Assay

The ALDEFLUOR assay was performed as described by the manufacturer (Stem Cell Technologies, Vancouver, British Columbia, Canada). Briefly, cells were harvested and resuspended in assay buffer at 1×10⁶ cells/ml and added to a tube containing 5 μl/ml of activated ALDEFLUOR substrate BAAA (BODIPY-aminoacetaldehyde). Half the sample was transferred to a tube containing the ALDH inhibitor diethylaminobenzaldehyde. Both samples were incubated at 37° C. for 30 minutes. The cells were resuspended in assay buffer and assayed on a FAC Scan II flow cytometer. The ALDH bright region was based on the control diethylaminobenzaldehyde sample that was gated to have less than five events.

### Mammosphere Generation

Culture cells were harvested in trypsin-EDTA and carefully resuspended in mammary epithelial growth medium (Lonza, Walkersville, Md.). Cells were filtered through sterile filters (BD Discovery Labware, Bedford, Mass.) to obtain a single cell suspension and plated at 5000 cells per well of a six-well ultra low-attachment plate (Corning, Lowell, Mass.). Mammospheres were assayed 3 to 10 days after cell plating. An average of five fields/well was counted using ImagePro software (Media Cybernetics, Bethesda, Md.), and experiments were done in quadruplicates.

### Immunohistochemistry

Immunohistochemistry was performed as described by earlier (Mironchik et al. (2005) Cancer Res 65, 10801-10809). Briefly, tumors were fixed overnight in 4% formalin and embedded in paraffin for sectioning. Antigen retrieval was carried out by boiling in sodium citrate for 10 minutes. Blocking was performed with normal goat serum. Sections were incubated overnight with 1:50 dilution of Twist primary antibody (in-house generated) and for 30 minutes with secondary antirabbit biotinylated antibody (Vector Laboratories, Burlingame, Calif.). Avidin-biotin mixture was added on the slides, followed by detection with DAB followed by counterstaining with hematoxylin. Sections were mounted using Permount (Thermo-Fisher Scientific, Waltham, Mass.) and photographed on a Nikon Eclipse 80i fluorescence microscope using a CoolSnap ES camera (Nikon Instruments).

### Promoter Analysis

Cloning of the ER promoter and the Twist deletion constructs has been described elsewhere (24, 25). The ER promoter constructs were transiently transfected (TransIT-LT1, Minis Bio Corporation, Madison, Wis.) along with the Twist expression constructs in the breast cancer cell line MCF-7. An enhanced green fluorescent protein expression plasmid pEGFP-N1 (Clontech, Mountain View, Calif.) was used to determine transfection efficiency (26).

The CD24 promoter (1600 bp) was polymerase chain reaction (PCR) amplified from human genomic DNA and cloned into the pGL4 vector upstream of the luciferase reporter (Promega, Madison, Wis.). Promoter assays were carried out in 24-well plates containing 50,000 cells. MCF-7 cells were transiently cotransfected with pGL4-CD24 reporter construct, pEFla-Twist plasmid, phRL-TK Renilla control plasmid, and pCR3.1 nonspecific plasmid. After transfection, the cells were incubated for varying times (12, 24, and 48 hours), assayed using the dual luciferase kit (Promega), and quantified on a luminometer (Berthold Sirius, Oak Ridge, Tenn.).

### Chromatin Immunoprecipitation

Chromatin immunoprecipitation (ChIP) was performed as described earlier (27). Briefly, MCF-7/Twist cells were cross-linked with formaldehyde and quenched with glycine. The cells were lysed to release chromatin, which was sheared by sonication to an average size of 500 to 1000 bp. The chromatin was precleared with preblocked, formalin-fixed, heat-killed Staphylococcus aureus cells. The positive control used was an anti-acetyl histone H3 antibody (Millipore, Billerica, Mass.), whereas the negative controls consisted of no-antibody and no-chromatin samples. The antibody-chromatin-Staph A complex was washed in stringent-buffered conditions and eluted from the complex, reverse cross-linked at high temperature, and phenol-chloroform extracted. The resulting purified DNA was PCR amplified using primers that flank the putative Twist binding sites within the CD24 promoter. The PCR primers (5′-TTGGGGTGGCAGCAGAAAGCATAG-3′ and 5′-AGGGATTCGGGAAGCAGCCAG TAG-3′) were designed for specific amplification of the ER promoter from −2078 to −1838 bp.

### Methylation Inhibitor and HDAC Inhibitor Treatment

Cells were treated with 1 μM AZA for three days and DNA processed for demethylation studies by methylation specific quantitative PCR (MS-qPCR). Total proteins were extracted and immunoblotted to analyze the effect of demethylation on ER expression. Cells were treated with HDAC inhibitor VPA at 10 μM concentration for three days and processed similarly.

### Animal Studies

Mice were anesthetized with acepromazine (62.5 mg/kg) and xylazine (6.5 mg/kg) or ketamine (100 mg/kg) and xylazine (10 mg/kg) in saline administered i.p. for xenograft implantation procedures. Mice were orthotopically injected in the breast with 2×10⁶ MCF-7/Twist or control MCF-7 cells in 100 μl sterile complete media in the second mammary fat pad. A total of 15 female mice were injected with MCF-7/Twist cells and five female mice were injected with control MCF-7 cells. Estradiol pellets were 90 day slow release (0.18 mg/pellet, Innovative Research of America, Sarasota, Fla.) and tamoxifen pellets were 60 day slow release (5 mg/pellet, IRA).

Five to ten millionMCF-7/Twist cells were labeled with CD44-FITC and CD24-PE antibodies as described earlier. Subsequently, the labeled cells were sorted into CD44⁺/CD24^{-/low} and CD44⁺/CD24⁺ subpopulations using a FACS Diva flow cytometer. The cells were cultured for one to two splittings before harvesting. The cells were trypsinized and resuspended with media/Matrigel (1:1) before orthotopically injecting various cell numbers in the mammary fat pad of 4- to 6-week-old female severe combined immunodeficient (SCID) mice (National Cancer Institute, Fredrick, Md.).

All animal experiments were done under Institutional Animal Care and Use Committee (IACUC) guidelines established at the Johns Hopkins University School of Medicine.

### Measurement of Vascular Volume (VV) and Permeability-surface Area Products (PS)

The measurement of VV and PS has been described in detail elsewhere (19, 28). Three-dimensional images were drawn for all the 17 mice (MCF-7 (+E2) n=5, MCF-7/Twist (+E2) n=6, MCF-7/Twist (−E2) n=6), and the images presented are representative of each group. The parameters were 8 slices, 1 mm slice thickness, FOV=32 mm, 8 scans, 0.25 mm in plane spatial resolution.

Twist and ER mRNA Expression Levels in Human Breast Cancers

Frozen breast cancer samples controlled for adequate tumor content (over 80%) by laser capture dissection were obtained from the University Medical Center, Utrecht, The Netherlands. Total RNA was isolated using Trizol, reverse transcribed using SuperScript III (Invitrogen, Carlsbad, Calif.), and quantitative real-time PCR amplified. Expression values were normalized with the 36B4 gene.

### Statistical Analysis

Data was analyzed by independent, two-sided Student's T-test. Statistics with respect to VV and PS were performed by Scheffe's test. We examined correlation in normalized (to normal breast tissue) expression between TWIST and ER genes, overall, and within each grade, using the non-parametric, Spearman rank test. For all analysis, P values below 0.05 were considered significant. In all figures, (*) denotes P<0.05, (**) denotes P<0.005, and (***) denotes P<0.0005.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for identifying a subject as having neoplasia having high metastatic potential, the method comprising:
a) obtaining a biological sample from a subject;
b) detecting the level of Twist and CD44 polypeptides or nucleic acid molecules, and the level of CD 24 polypeptides or nucleic acid molecules in the biological sample from the subject;
c) analyzing the relative levels of Twist and CD44 polypeptides or nucleic acid molecules, and the level of CD 24 polypeptides or nucleic acid molecules in the biological sample to levels present in a control;
d) identifying the neoplasia as having high metastatic potential when an increase in the level of Twist and CD44 polypeptides or nucleic acid molecules, and a reduction in the level of CD 24 polypeptides or nucleic acid molecules in a biological sample from the subject is detected.

2. (canceled)

3. The method of claim 1 wherein the neoplasia is an epithelial carcinoma selected from the group consisting of breast cancer, prostate cancer, lung cancer, brain cancer, ovarian cancer, or any other cancer characterized by high Twist expression.

4.-5. (canceled)

6. The method of claim 1, wherein the method further comprises detecting one or more polypeptide or polynucleotide biomarkers selected from the group consisting of ABCC1, ABCG2, ALDH, CD10, CD133, choline kinase, CXCR4, lactate dehydrogenase, Lin negative, PROCR, ER, ESA, MUC1, and p27.

7. The method of claim 1, wherein the biological sample is a liquid or tissue sample.

8. The method of claim 7, wherein the liquid sample is a nipple aspirant, ductal lavage specimen, or milk.

9.-10. (canceled)

11. The method of claim 1, wherein the sample is further characterized for one or more of level of estrogen receptor expression, level of estrogen receptor promoter methylation, or level of estrogen receptor promoter histone deacetylation.

12. A method of selecting a treatment for a subject identified as having breast cancer or ductal carcinoma in situ, the method comprising:
a) obtaining a biological sample from a subject;
b) detecting the level of Twist and CD44 polypeptides or nucleic acid molecules, and the level of CD 24 polypeptides or nucleic acid molecules in the biological sample from the subject;
c) analyzing the relative levels of Twist and CD44 polypeptides or nucleic acid molecules, and the level of CD 24 polypeptides or nucleic acid molecules in the biological sample to levels present in a control;
d) identifying the neoplasia as indicative that aggressive therapy should be selected when an increase in the level of Twist and CD44 polypeptides or nucleic acid molecules, and a reduction in the level of CD 24 polypeptides or nucleic acid molecules in a biological sample from the subject is detected and the presence of Twist⁺/CD44⁺/CD24^{+/low} cells is detected.
detecting an increase in the level of Twist and CD44 polypeptides or nucleic acid molecules, and a reduction in the level of CD24 polypeptides or nucleic acid molecules in a biological sample from the subject relative to levels present in a control, wherein the levels are indicative of the therapy to be selected.

13.-14. (canceled)

15. The method of claim 1, wherein the method further comprises detecting estrogen receptor (ER), a human epidermal growth factor receptor 2 (Her2), and/or a progesterone receptor (PR) in the sample.

16. A method for treating a subject identified as having a breast cancer or ductal carcinoma in situ that comprises Twist⁺/CD44⁺/CD24″^{/low} cells, the method comprising administering to the subject a combination of Twist inhibitory nucleic acid molecule, a methylation inhibitor, and an HDAC inhibitor.

17. The method of claim 16, wherein the Twist inhibitory nucleic acid molecule is an siRNA, shRNA, or antisense RNA.

18. The method of claim 16, wherein the methylation inhibitor is selected from the group consisting of 5-azacytidine, 5-azadeoxycytidine, procainamide, zebularine, and RG108.

19. The method of claim 16, wherein the HDAC inhibitor is selected from the group consisting of valproic acid, sodium butyrate, and Trichostatin A.

20. The method of claim 16, wherein the method increases estrogen receptor expression.

21.-24. (canceled)

25. The method of claim 1, wherein the polypeptide is detected by a method selected from the group consisting of an ELISA, immunocytochemistry, immunohistochemistry, flow cytometric analysis, radioimmunoassay, Western blot, and mass spectrometry

26. The method of claim 1, wherein the nucleic acid molecule is detected by a method selected from the group consisting of PCR, rtPCR, quantitative rtPCR, using a probe that hybridizes to the nucleic acid molecule, and microarray analysis.

27.-34. (canceled)

35. A kit for the detection of a Twist⁺/CD44⁺/CD24″^{/low} cell, the kit comprising reagents capable of specifically hybridizing or binding to Twist, CD44, and CD24 nucleic acid molecules or polypeptides.
